(19) United States
(12) Patent Application Publication
(10) Pub. No.: US 2025/0213519 A1
(43) Pub. Date: Jul. 3, 2025

(54) SYNERGY FOR INCREASING INSULIN SENSITIVITY

(71) Applicant: Board of Supervisors of Louisiana State University and Agricultural and Mechanical College, Baton Rouge, LA (US)

(72) Inventors: Frank Greenway, Baton Rouge, LA (US); Ann Coulter, Baton Rouge, LA (US); Candida Rebello, Baton Rouge, LA (US); Guru Ramanathan, Baton Rouge, LA (US)

(21) Appl. No.: 18/852,280

(22) PCT Filed: Mar. 28, 2023
(86) PCT No.: PCT/US2023/016541

§ 371 Date: Sep. 27, 2024

Related U.S. Application Data
(60) Provisional application No. 63/324,515, filed on Mar. 28, 2022.

(51) Int. Cl. A61K 31/352; A61K 31/015; A61K 45/06; A61P 3/04

(52) U.S. Cl. CPC A61K 31/352; A61K 31/015; A61K 45/06; A61P 3/04

(57) ABSTRACT

The invention is directed to compositions and methods for treating metabolic disorders.

This application is an International Application which claims priority from U.S. Provisional Patent Application No. 63/324,515, filed on Mar. 28, 2022, the contents of which are incorporated by reference herein in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Obesity and overweight now affects over half of the adult United States Population and an increase in the prevalence of type 2 diabetes follows the prevalence of obesity by about a decade. Not only is the increasing obesity pandemic fueling an increase in type 2 diabetes, but a treatment for type 2 diabetes that causes weight loss is useful in combating these two health problems and improving public health.

### SUMMARY OF THE INVENTION

Aspects of the invention are drawn towards a composition comprising a therapeutically effective amount of at least one flavanone and a therapeutically effective amount of at least one flavonol. For example, the flavanone is naringenin. For example, the flavonol is myricetin.

In embodiments, the composition further comprises a therapeutically effective amount of at least one carotenoid. For example, the at least one carotenoid is selected from the group consisting of beta carotene, lycopene, or lutein.

In embodiments, the flavanone is naringenin, wherein the flavonol is myricetin, and wherein the carotenoid is beta carotene.

In embodiments, the composition further comprises a sufficient amount of a pharmaceutically acceptable carrier.

In embodiments, the composition comprises about 150 mg to about 900 mg of at least one flavanone.

In embodiments, the composition comprises about 1 mg to about 100 mg of at least one flavonol.

In embodiments, the composition comprises about 1 mg to about 12 mg of at least one carotenoid.

In embodiments, the composition further comprises one or more additional active agents. For example, the additional active agent comprises an anti-obesity agent.

In embodiments, the composition is provided as an injectable solution, an oral dose, a topical cream, a topical gel, or a medical food.

Aspects of the invention are also directed to a method for local fat reduction. In embodiments, the method comprises administering to a site on a subject a therapeutically effective amount of the composition as described herein.

Further, aspects of the invention are drawn towards a method for treating a subject afflicted with a metabolic disorder. For example, the metabolic disorder comprises obesity or an insulin resistance disease, such as type 2 diabetes, polycystic ovarian syndrome, nonalcoholic steatohepatitis, or metabolic syndrome.

In embodiments, the method comprises administering to a subject a therapeutically effective amount of the composition as described herein.

### BRIEF DESCRIPTION OF THE FIGURES

FIG. 1 shows UCP1 and GLUT4 mRNA expression in human adipocyte cultures treated with CDK5-inhibitor flavonols combined with naringenin. Human adipocyte cultures from individuals with obesity were treated for seven days with 8 μM naringenin extract (nar) and 2.5 μM myricetin, 2.5 μM quercetin or 2.5 μM kaempferol.

FIG. 2 shows synergistic gene expression effects of myricetin with naringenin in human adipocytes. Mature human adipocytes derived from the stromal vascular fraction of adult adipose tissue from individuals with obesity and diabetes were differentiated in culture and treated for seven days with 8 μM naringenin (in Citrus sinensis extract) and 2.5 μM myricetin. C=vehicle control, nar=naringenin extract and myr=myricetin, *represents p<0.05 by anova single factor analysis.

FIG. 3 shows concentration-dependence of myricetin and naringenin effects on gene expression in human adipocytes. Panel a and panel b: Naringenin concentration curve with constant concentration of myricetin (3 μM myr). Panel a shows UCP1 mRNA levels, and panel b shows GLUT4 mRNA. Naringenin (nar) was added at concentrations ranging from 0.03 μM to 8 μM. Addition of naringenin increased gene expression in a concentration-dependent manner. Panel c and panel d: Myricetin concentration curve with constant naringenin concentration (8 μM nar). Panel c shows UCP1 mRNA levels, and panel d shows GLUT4 mRNA levels. Adipocytes were treated for seven days with 8 μM naringenin extract (nar). Myricetin (myr) was added at concentrations ranging from 0.1 μM to 6 μM as shown.

FIG. 4 shows adipocytes treated with 8 μM naringenin ext (ne) and 5 nM to 50 nM myricetin (myr).

FIG. 5 shows upregulation of metabolic genes by naringenin and β-carotene with myricetin (rym). Top panel: Naringenin and β-carotene synergistically induce gene expression. Differentiated cultures of human adipocytes from overweight or obese donors were treated for 7 days with 8 μM naringenin extract (nar) and 2 μM βC as indicated. (* indicates statistical synergy). Bottom panel: Myricetin (50 nM) enhances the effects on gene expression by nar +βC (NRβC).

FIG. 6 shows UCP-1 mRNA expression after 7 days of exposure to Naringenin Orange extract.

FIG. 7 shows UCP-1 mRNA expression in adult abdominal fat explants from 3 donors.

FIG. 8 shows naringenin increases the metabolic rate of human fat cells.

FIG. 9 shows adiponectin mRNA is increased in a manner similar to UCP-1.

FIG. 10 shows protein levels of UCP-1 and adiponectin went progressively over 7 days of 8 μM.

FIG. 11 shows ingestion of naringenin 150 mg three times a day in orange extract capsules increased metabolic rate by 3%. The blue line represents metabolic rate at the beginning of the study before the fat had a chance to increase the UCP-1. The green line is what her metabolic rate looked like after 8 weeks. The orange line subtracts the blue line from the green line to give the actual increase of metabolic rate from baseline compared to the end of the study.

FIG. 12 shows synergistic increases in expression of genes for thermogenesis and insulin sensitivity were observed after treating human adipocytes with 8 μM naringenin in orange extract and 2 μM β-carotene for 7 days.

FIG. 13 shows the flavonoid myricetin (rym) was added to 8 μM nar ext at concentrations ranging from 0.1 μM to 6 μM. The lowest concentration tested (0.1 μM) had the strongest activity for enhancing the activity of nar ext for induction of UCP1 expression.

FIG. 14 shows myricetin (Rym) was the most active at 50 nM with the combination of naringenin extract and β-carotene.

FIG. 15 provides mRNA data levels after 7 days of treatment, which shows that combining myricetin with NR does not upregulate SIRT1 or SIRT3 mRNAs compared to NR alone.

FIG. 16 provides Western blot data of SIRT3 protein levels, which shows that adding myricetin to NR does not increase SIRT3 protein levels.

FIG. 17 provides Western blot data of PGC-1α protein levels, which shows that myricetin and NR do not induce PGC-1α compared to NR or myricetin alone.

FIG. 18 shows PPARγ-Ser-273 levels after NRBC treatment.

FIG. 19 shows data depicting that the addition of myricetin to NRBC did not further decrease PPARg P-273 relative to NRBC.

FIG. 20 shows pro-vitamin A carotenoids and NR act synergistically to elevate levels of UCP1 mRNA. Adipocytes from 4 donors with obesity were treated for seven days with vehicle (control) or 8 μM NR and 2 μM carotenoids. Panel A: NR+BC; Panel B: NR+Lutein; Panel C: NR+Lycopene mRNA levels were measured using quantitative RTPCR. Data is expressed in least squares means±standard error. Synergy for mRNA was calculated as: sum of differences ((NR−Control)+(BC−Control)) vs (NRBC−Control). *p<0.001 for synergy, sum of differences versus NRBC. NR: naringenin BC: beta carotene.

FIG. 21 shows NR and BC synergistically induce metabolism genes. Panel A provides mRNA levels. Panel B provides Western Blots of Protein levels. Panel C provides shows protein levels were measured with β-actin as loading control. Adipocytes from three or more donors with obesity were treated for seven days. mRNA data are expressed as least squares means±standard error. Synergy for mRNA was calculated as: sum of differences ((NR−Control)+(BC−Control)) vs (NRBC−Control). *p<0.05 for synergy. NR: Naringenin BC: beta carotene NRBC: naringenin and beta carotene

FIG. 22 shows NRBC upregulates a subset of key regulatory proteins without mRNA increases. Panel A provides data for PPARα. Panel B provides data for PPARγ. Panel C provides data for PGC-1α. Panel D provides data for NAMPT. Adipocytes from three to five donors with obesity were treated for seven days. Protein was measured by Western Blotting with β-actin used to adjust for loading. mRNA was quantified by real-time PCR. Data are expressed as least squares means±standard error, *p<0.001 NR: Naringenin BC: beta carotene NRBC: naringenin and beta carotene

FIG. 23 shows whole transcriptome sequencing analysis of pathways stimulated by naringenin and β-carotene (NRBC). Adipocytes from two donors with BMI of 27 and 36 kg/m2 were treated with cell medium (vehicle control) or NRBC for seven days. cDNA libraries from expressed transcripts were constructed, sequenced and differential gene expression was analyzed. Gene-sets with false discovery rate (FDR)≤5% were considered as significantly enriched, and the top pathways are shown.

FIG. 24 shows RT-PCR validation of genes upregulated by naringenin and beta carotene (NRBC) in RNA sequencing analysis. Adipocytes from three donors with obesity were treated for seven days. mRNA levels were measured using quantitative RT-PCR. Data are expressed as mean #standard error, *p<0.001.

FIG. 25 shows relative receptor levels in white adipocytes (untreated) and NRBC-treated cells. Panel A shows data for low abundance. Panel B shows data for high abundance. Adipocytes from two donors with obesity were treated with cell medium (Control) or NRBC for seven days and transcript sequencing analysis was conducted. Data are expressed as mean normalized transcript counts (DEseq2). * indicates Padj<0.002 for Control vs NRBC. β-adrenergic receptors (β1AR, β2AR, β3AR), G-protein coupled bile acid receptor (TGR5), Transient receptor potential cation channel subfamily M member 8 (TRPM8), Melanocortin-1 receptor (MC1R), Adenosine receptors A1 and A2B (ADORA1, ADORA2B), Natriuretic peptide receptors (NPR1, NPR3), G-protein coupled estrogen receptor 1 (GPER1), Parathyroid hormone receptor 1 (PTHR1), Growth hormone receptor (GHR).

FIG. 26 shows hormone-stimulated lipolysis in white adipocytes (untreated) and NRBC-pretreated cells. After 7d pretreatment with vehicle (untreated) or NRBC, adipocytes were exposed for 4.5 hours to receptor agonists in KRB buffer. Supernatants were removed for measurement of glycerol. Data are presented as least squares mean±standard error from experiments using cells from four different donors with BMIs ranging from 27 to 36, each with at least 6 replicates. (*indicates a difference between untreated white adipocytes and NRBC-treated adipocytes p≤0.02) cAMP 8-Cpt-CAMP 200 μM, ANP atrial natriuretic peptide 0.1 μM, PTH parathyroid hormone (1-34) 1 μM, isoprot isoproterenol 1 μM, dobutam dobutamine 1 μM, estradiol 1 μM, GH growth hormone 250 ng/ml, ACTH adrenocorticotropin hormone 1 μM, CDCA chenodeoxycholic acid 30 μM, adenosine 1 μM, menthol 100 μM.

FIG. 27 shows analysis of RXR isoforms bound to immunoprecipitated PPARγ complexes. Adipocytes were treated with vehicle or NRBC for seven days. RXRγ, RXRα and PPARγ protein levels were analyzed by Western blotting. For analysis of whole cell protein levels (input), 50 μg of protein lysate was loaded in each lane. PPARγ was immunoprecipitated from 600 μg of the same whole cell lysate for analysis of PPARγ cofactor binding. An anti-IgG light chain secondary antibody was used for detection. This experiment was repeated three times with adipocytes from obese donors.

FIG. 28 provides a schematic showing the paradigm for remodeling of white adipocytes by NRBC. NR and BC bind nuclear receptors and activate gene expression at PPRE motifs. UCP1 and other uncoupling compounds and mitochondrial proteins mediate multiple energy-wasting enzymatic cycles that generate heat. PM20D1 regulates synthesis and degradation of N-acyl amino acids (NAA), molecules that directly uncouple mitochondria and increase energy expenditure. The synthesis and breakdown of creatine phosphate by the mitochondrial creatine kinases CKMT1A, CKMT1B and CKMT2 facilitates ATP-coupled respiration and enhances oxygen consumption. PDK4 directs pyruvate into synthesis of glycerol, fatty acids, and TGs to promote futile TG recycling. Lipolytic receptors and PKA are upregulated, increasing responsiveness to hormones and lipolysis. Fatty acids are transferred into mitochondria to fuel thermogenesis. Genes are turned on for production of bioactive peptides and lipokines which have autocrine insulin sensitizing effects and are secreted into circulation. NR (Naringenin), BC (β-carotene) Created with Biorender.com

FIG. 29 shows NR and BC synergistically induce metabolism proteins. Uncropped Western Blots of proteins provided in FIG. 21. Adipocytes from donors with obesity with obesity were treated for seven days. NR (Naringenin), BC (β-carotene), NRBC (Naringenin and β-carotene)

FIG. 30 shows NRBC upregulates a subset of key regulatory proteins without mRNA increases. Uncropped Western Blots of proteins provided in FIG. 22. Adipocytes from donors with obesity with obesity were treated for seven days. NR (Naringenin), BC (β-carotene), NRBC (Naringenin and β-carotene)

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

In order that the invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

The phraseology or terminology in this disclosure is for the purpose of description and not of limitation, such that the terminology or phraseology of the present specification is to be interpreted by the skilled artisan in light of the teachings and guidance.

The singular forms “a,” “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Wherever any of the phrases “for example,” “such as,” “including” and the like are used herein, the phrase “and without limitation” is understood to follow unless explicitly stated otherwise. Similarly, “an example,” “exemplary” and the like are understood to be non-limiting.

The term “substantially” can refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” or “approximately” can refer to within an acceptable error range for the value as determined by one of ordinary skill in the art, which will depend in part on how the value is measured or determined, i.e., the limitations of the measurement system. For example, “about” can refer to within 3 or more than 3 standard deviations, per the practice in the art. Alternatively, “about” can refer to a range of up to 20%, e.g., up to 10%, up to 5%, or up to 1% of a given value. Alternatively, with respect to biological systems or processes, the term can refer to within an order of magnitude, e.g., within 5-fold, or within 2-fold, of a value.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The “median” value can refer to the median value obtained from a population of subjects having a cancer. The median values can be previously determined reference values or can be contemporaneously determined values.

It will be understood that, although the terms first, second, etc. can be used herein to describe various elements, these elements cannot be limited by these terms. These terms are only used to distinguish one element from another. For example, a first subject can be termed a second subject, and, similarly, a second subject can be termed a first subject, without departing from the scope of the present disclosure. The first subject and the second subject are both subjects, but they are not the same subject. Furthermore, the terms “subject,” “user,” and “patient” are used interchangeably herein.

The term “consisting essentially of” can refer to a composition, whose only active ingredient is the indicated active ingredient, however, other compounds can be included which are for stabilizing, preserving, etc. the formulation, but are not involved directly in the therapeutic effect of the indicated active ingredient. In some embodiments, the term “consisting essentially of” can refer to components which facilitate the release of the active ingredient. For example, a composition described herein can consist essentially of at least one flavanone and at least one flavonol. Such composition can also other compounds which are for stabilizing, preserving, or facilitating the release of the formulation, but are not involved directly in the therapeutic effect of the indicated active ingredient.

The term “consisting” can refer to a composition, which contains the active ingredient and a pharmaceutically acceptable carrier or excipient. For example, a composition described herein can consist of at least one flavanone and at least one flavonol, together with a pharmaceutically acceptable carrier or excipient.

Unless defined otherwise, all technical and scientific terms used herein have the meaning commonly understood by a person skilled in the art to which this invention belongs. The following references provide one of ordinary skill in the art with a general definition of many of the terms used herein: Singleton et al, Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991); Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al, ed., 1994); “A Practical Guide to Molecular Cloning” (Perbal Bernard V., 1988); “Phage Display: A Laboratory Manual” (Barbas et al, 2001). The contents of these references and other references containing standard protocols, widely known to and relied upon by those of skill in the art, including manufacturers' instructions are hereby incorporated by reference as part of the presently disclosed subject matter.

Various exemplary embodiments of the invention described herein can comprise compositions and methods for treating a subject afflicted with a metabolic disorder or preventing the onset of a metabolic disorder, such as obesity, insulin resistance, type 2 diabetes, and non-alcoholic steohepatis. For example, embodiments can comprise administering to the subject a composition comprising a therapeutically effective amount of at least one flavanone and at least one flavonol. In embodiments, the composition can further comprise at least one carotenoid. In embodiments, the composition can be applied to the skin of a subject as a topical cream or gel, is ingested by the subject as a food or an oral dose, or is injected in the form of an injectable solution.

In embodiments, the composition comprises a therapeutically effective amount of at least one active agent, such as at least one flavanone and at least one flavonol. The term “active agent” can refer to a compound as described herein, such as at least one flavanone and/or at least one flavonol. For example, one or more active ingredients can be accountable for the intended biological effect (i.e., for treatment or prevention of a metabolic disease).

In embodiments, the composition can optionally comprise at least one additional active agent, such as at least one carotenoid. In embodiments, the one or more additional active agents can further comprise an anti-obesity agent, such as phentermine, and other agents described herein. The phrase “additional active agent” can refer to an agent, other than a compound(s) of the inventive composition, that exerts a pharmacological, or any other beneficial activity.

Those skilled in the art will recognize, or be able to ascertain, using no more than routine experimentation, numerous equivalents to the specific compositions and methods described herein. Such equivalents are considered to be within the scope of this invention, and are covered by the following claims.

Aspects of the disclosure are drawn to a composition comprising a therapeutically effective amount of at least one flavanone and at least one flavonol, optionally comprising at least one carotenoid.

As used herein, a “therapeutic composition” can refer to a composition comprising one or more active ingredient(s) required to cause an effect when an effective amount of the composition is administered to a subject in need thereof. For example, the effect can be prevention or treatment of a metabolic disorder or local fat reduction.

In embodiments, the composition can comprise a therapeutically effective amount of at least one flavanone. For example, the flavanone can be naringenin. Naringenin is a flavanone, a type of flavonoid, that is flavorless and colorless. It is the predominant flavanone in grapefruit, and is found in a variety of fruits and herbs. Naringenin has the skeleton structure of a flavanone with three hydroxy groups at the 4′, 5, and 7 carbons. It can be found both in the aglycol form, naringenin, or in its glycosidic form, naringin, which has the addition of the disaccharide neohesperidose attached via a glycosidic linkage at carbon 7. Naringenin and its glycoside has been found in a variety of herbs and fruits, including grapefruit, bergamot, sour orange, tart cherries, tomatoes, cocoa, Greek oregano, water mint, drynaria as well as in beans. Ratios of naringenin to naringin vary among sources, as do enantiomeric ratios. The isolation methods of naringenin are well known in the art. See, for example, Wang, Chung-Yi, et al. “Quality changes in high hydrostatic pressure and thermal pasteurized grapefruit juice during cold storage.” Journal of food science and technology 55.12 (2018): 5115-5122.

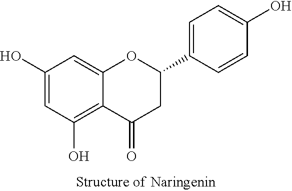 [SMILES: O=C1C[C@@H](c2ccc(O)cc2)Oc2cc(O)cc(O)c21]

Embodiments can further comprise a therapeutically effective amount of at least one flavonol. Flavonols are a class of flavonoids that are present in a wide variety of fruits and vegetables. The backbone of a flavonol and its substituent numbers are:

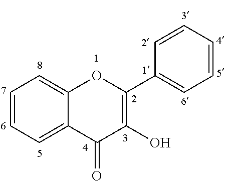 [SMILES: O=c1c(O)c(-c2ccccc2)oc2ccccc12]

Non-limiting examples of flavonols include 3-hydroxyflavone, azaleatin, fisetin, galangin, gossypetin, kaempferide, kaempferol, isorhamnetin, morin, myricetin, natsudaidain, pachypodol, quercetin, rhamnazin, and rhamnetin.

Myricetin is a flavonoid that can be found in many common foods such as berries, vegetables, fruits, tea and red wine. It is extracted from all parts of the Chinese bayberry tree Myrica Rubra in Japan where it is used to flavor snack foods and drinks. It is listed as GRAS (generally recognized as safe) by Flavor and Extract Manufacturer Association and was recently judged to be safe at current estimated dietary exposures by the Joint Food and Agriculture Organization of the United Nations (FAO)/World Health Organization (WHO) Expert Committee on Food Additives (JECFA). Specifications related to the identity and purity of myricetin for use as a flavoring agent have been established by JECFA.

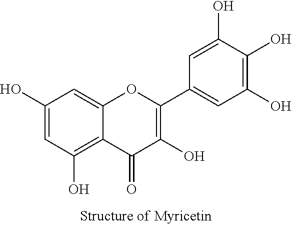 [SMILES: O=c1c(O)c(-c2cc(O)c(O)c(O)c2)oc2cc(O)cc(O)c12]

Quercetin is a flavonol found in fruits, vegetables, leaves, seeds, and grains. For example, quercetin can be found in capers, lovage leaves, buckwheat seeds, radish leaves, dill, cilantro, fennel leaves, red onion, radicchio, watercress, kale, chokeberry, bog blueberry, cranberry, lingonberry, and black plums.

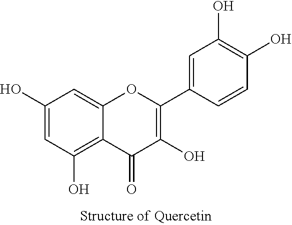 [SMILES: O=c1c(O)c(-c2ccc(O)c(O)c2)oc2cc(O)cc(O)c12]

Kaempherol is a natural flavonol found in a variety of plants and plant-derived foods. Common foods that contain kaempferol include apples, grapes, tomatoes, green tea, potatoes, onions, broccoli, Brussels sprouts, squash, cucumbers, lettuce, green beans, peaches, blackberries, raspberries, and spinach. Plants that are known to contain kaempferol include Aloe vera, Coccinia grandis, Cuscuta chinensis, Euphorbia pekinensis, Glycine max, Hypericum perforatum, Pinus sylvestris, Moringa oleifera, Rosmarinus officinalis, Sambucus nigra, and Toona sinensis, and Ilex. It also is present in endive.

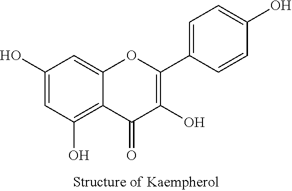 [SMILES: O=c1c(O)c(-c2ccc(O)cc2)oc2cc(O)cc(O)c12]

Embodiments can further comprise a therapeutically effective amount of at least one carotenoid, such as beta-carotene, leutine, or lycopene. Carotenoids are yellow, orange, and red organic pigments that are produced by plants and algae, as well as several bacteria and fungi. [here are over 1,100 known carotenoids which can be categorized into two classes, xanthophylls (which contain oxygen) and carotenes (which are purely hydrocarbons, and contain no oxygen). Carotenoids can be derivatives of tetraterpenes, meaning that they are produced from 8 isoprene molecules and contain 40 carbon atoms. Carotenoids absorb wavelengths ranging from 400-550 nanometers (violet to green light). This causes the compounds to be deeply colored yellow, orange, or red. The structure of carotenoids imparts biological abilities, including photosynthesis, photoprotection, plant coloration, and cell signaling. The structure of the carotenoid is a polyene chain consisting of 9-11 double bonds and, without wishing to be bound by theory, terminating in rings. This structure of conjugated double bonds leads to a high reducing potential, or the ability to transfer electrons throughout the molecule.

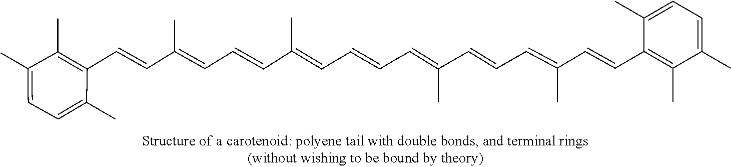

The isolation methods of carotenoids are well known in the art. See, for example, Vieira, Flávia A., and Sónia PM Ventura. “Efficient Extraction of Carotenoids from Sargassum muticum Using Aqueous Solutions of Tween 20.” Marine drugs 17.5 (2019): 310.

Lutein is a xanthophyll and one of 600 known naturally occurring carotenoids. Lutein is synthesized only by plants and like other xanthophylls is found in high quantities in green leafy vegetables such as spinach, kale and yellow carrots.

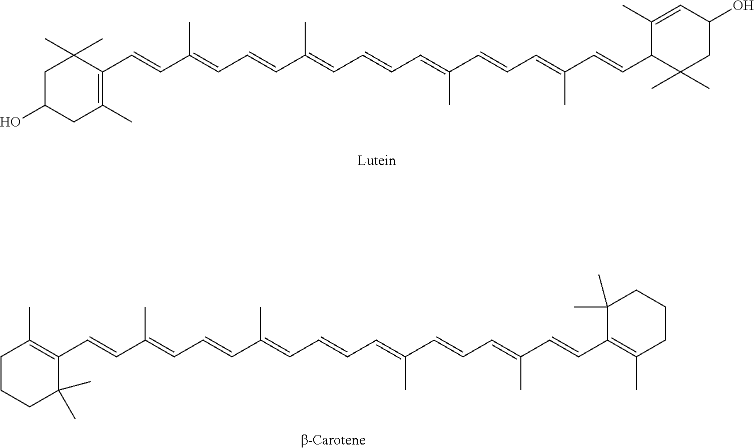

Lycopene is a bright red carotenoid hydrocarbon found in tomatoes and other red fruits and vegetables, such as red carrots, watermelons, gac melons, and papayas, but it is not present in strawberries or cherries. Although lycopene is chemically a carotene, it has no vitamin A activity. Foods that are not red can also contain lycopene, such as asparagus and parsley.

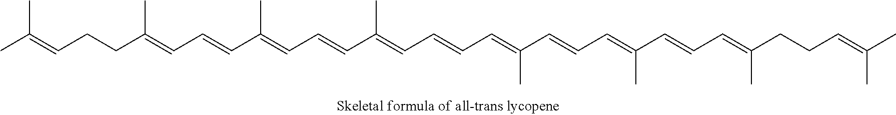 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

β-Carotene is an organic, strongly colored red-orange pigment abundant in plants and fruits. It is a member of the carotenes, which are terpenoids (isoprenoids), synthesized biochemically from eight isoprene units and thus having 40 carbons. Among the carotenes, β-carotene is distinguished by having beta-rings at both ends of the molecule. β-Carotene is biosynthesized from geranylgeranyl pyrophosphate. β-Carotene is the most common form of carotene in plants. In nature, β-carotene is a precursor (inactive form) to vitamin A via the action of beta-carotene 15,15′-monooxygenase.

As described herein, aspects of the disclosure are drawn to compositions comprising therapeutically effective amounts of at least one flavanone and at least one flavonol, optionally comprising at least one carotenoid. Further, aspects of the disclosure are drawn to methods comprising therapeutically effective amounts of a composition comprising at least one flavanone and at least one flavonol, optionally comprising at least one carotenoid. The term “therapeutically effective amount” can refer to those amounts that, when administered to a subject in view of the nature and severity of that subject's disease or condition, will have a therapeutic effect, e.g., an amount which will cure, prevent, inhibit, reduce or at least partially arrest or partially prevent a target disease or condition. In some embodiments, the term “therapeutically effective amount” or “effective amount” can refer to an amount of a therapeutic agent that when administered alone or in combination with an additional therapeutic agent to a cell, tissue, or subject is effective to prevent or ameliorate the disease or condition, such as a metabolic disorder, or the progression of the disease or condition. A therapeutically effective dose further refers to that amount of the therapeutic agent sufficient to result in amelioration of symptoms, e.g., treatment, healing, prevention, reduction or amelioration of the relevant medical condition, such as local fat reduction, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

The dosage can vary depending upon a number of factors known to those of ordinary skill in the art. For example, the dose(s) can vary depending upon the identity, age, sex, health, weight, size, and condition of the subject or sample being treated, and the nature and extent of the condition. The dosage can further depend on the effect which is required by the practitioner, pharmacodynamic characteristics of the active ingredient and its mode and route of administration; time of administration of active ingredient; kind of concurrent treatment, frequency of treatment and the effect; and rate of excretion. These amounts can be readily determined by the skilled artisan.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage can vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Depending on the severity of the condition and the responsiveness of the subject to treatment, dosing can be of a single or a plurality of administrations, with course of treatment lasting from several days to several weeks, several months or several years, or until cure is effected or diminution of the disease state is achieved. Alternatively, the compositions are administered in order to prevent occurrence of a metabolic disorder in a subject at risk of developing a metabolic disorder. The compositions can be administered for prolonged periods of time (e.g. several days, several weeks, several months or several years) as to prevent occurrence of a metabolic disorder.

In some embodiments, the therapeutically effective amount of the carotenoid, such as β-carotene, lycopene, or lutein, can comprise less than about 1 mg/day, about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 21 mg/day, about 22 mg/day, about 23 mg/day, about 24 mg/day, about 25 mg/day, or greater than 25 mg/day. For example, the therapeutically effective amount of β-carotene comprises about 12 mg/day or less than about 12 mg/day.

In some embodiments, the therapeutically effective amount of the flavanone, such as naringenin, can comprise about 10 mg/day, about 50 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1100 mg/day, about 1200 mg/day, about 1300 mg/day, about 1400 mg/day, about 1500 mg/day, about 1600 mg/day, about 1700 mg/day, about 1800 mg/day, about 1900 mg/day, about 2000 mg/day, about 2500 mg/day, about 3000 mg/day, about 3500 mg/day, about 4000 mg/day, about 4500 mg/day, about 5000 mg/day, or greater than about 5000 mg/day. For example, the therapeutically effective amount of naringenin is between about 150 mg/day to about 900 mg/day.

In some embodiments, the therapeutically effective amount of the flavonol, such as myricetin, can comprise about 1 mg/day, about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

In embodiments, the therapeutically effective amount of a compound and/or composition can be administered once a day, twice a day, three times a day, or more, as needed.

According to an embodiment of the disclosure, the compositions can be administered at least once a day. For example, the compositions can be administered daily. According to another embodiment, the compositions can be administered twice a day, three times a day or more. In embodiments, the compositions can be administered weekly, such as about once a week or about twice a week. In embodiments, the compositions can be administered monthly, such as about once a month or about twice a month.

According to an embodiment of the disclosure, the composition can be administered to a subject chronically. Such is the case, for example, when treating a subject afflicted with a chronic disease or condition. In other embodiments, the composition can be administered to a subject as long as the subject is at risk of a disease or condition, or as long as symptoms of a disease or condition persists. For example, embodiments of the invention can be administered to a subject for at least 7 days, at least 10 days, at least 12 days, at least 14 days, at least 16 days, at least 18 days, at least 21 days, at least 24 days, at least 27 days, at least 30 days, at least 60 days, at least 90 days, or longer than 90 days.

Compounds, for example at least one flavanone and at least one flavonol, optionally comprising at least one carotenoid, can be incorporated into pharmaceutical compositions suitable for administration to a subject. The term “pharmaceutical composition” can refer to a therapeutically effective amount of a compound and/or composition described herein, with a pharmaceutically acceptable carrier or diluent. Such pharmaceutical compositions can comprise at least one flavanone and at least one flavonol, optionally comprising at least one carotenoid, and a pharmaceutically acceptable carrier or excipient. Thus, in some embodiments, the compounds of the invention are present in a pharmaceutical composition.

The phrases “physiologically acceptable carrier” and “pharmaceutically acceptable carrier” which can be interchangeably used can refer to a carrier, excipient, or diluent that does not cause significant irritation to the subject and does not abrogate the biological activity and properties of the administered active ingredients. An adjuvant is included under these phrases.

A pharmaceutically acceptable carrier can comprise one or more solvents, dispersion medias, coatings, antibacterial and antifungal agents, isotonic and absorption delaying agents, and the like, compatible with pharmaceutical administration to a subject. The use of such media and agents for pharmaceutically active substances is well known in the art. Any conventional media or agent that is compatible with the active compound can be used. Supplementary active compounds can also be incorporated into the compositions.

The term “excipient” can refer to an inert substance added to the composition (pharmaceutical composition) to further facilitate administration of an active ingredient of the present invention.

Techniques for formulation and administration of drugs can be found in “Remington's Pharmaceutical Sciences,” Mack Publishing Co., Easton, Pa., latest edition, which is incorporated herein by reference.

Compositions of the disclosure can be manufactured by processes well known in the art, e.g., by means of conventional mixing, dissolving, granulating, dragee-making, levigating, emulsifying, encapsulating, entrapping or lyophilizing processes.

Compositions for use in accordance with embodiments of the invention thus can be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Any of the therapeutic applications or methods of use described herein can be applied to any subject in need of such therapy, including, for example, a mammal such as a mouse, a rat, a dog, a cat, a cow, a horse, a rabbit, a monkey, a pig, a sheep, a goat, or a human. In some embodiments, the subject is a mouse, rat or human. In some embodiments, the subject is a mouse. In some embodiments, the subject is a rat. In some embodiments, the subject is a human.

A pharmaceutical composition of the invention can be formulated to be compatible with its intended route of administration. Examples of routes of administration include parenteral, e.g., intravenous, intradermal, subcutaneous, oral (i.e., capsule or medical food), nasal (e.g., inhalation), transdermal (topical, such as a cream), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersions. For intravenous administration, suitable carriers can include physiological saline, bacteriostatic water, Cremophor EM™ (BASF, Parsippany, N.J.) or phosphate buffered saline (PBS). The composition can be sterile and can be fluid to the extent that easy syringability exists. In embodiments, the composition can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules, compressed into tablets, or prepared as a medical food. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

In embodiments, the composition can be prepared as a medical food, dietary item or food ingredient. The term “dietary item” can include any product that undergoes at least one processing or culinary step prior to distribution and is consumed by a subject. Non-limiting examples of processing and culinary steps include mixing, cooking, baking, heating, chopping, chilling, freezing, packaging, canning, bagging, and storing. Non-limiting examples of dietary items include food products, dietary ingredients, medical foods, functional foods, beverages, dietary supplements, vitamins, minerals, and combinations thereof. Unprocessed, raw, or fresh foods, such as fresh fruits and vegetables, are not included herein within this term.

The term “food ingredient” can refer to any edible substance that is combined is with other edible substances, where the final combination is consumed as a food. The term “medical food” herein is defined by statute in the United States of America, Orphan Drug Act, section 5(b) (21 U.S.C. 360ee (b)(3)), which defines “medical food” as “a food which is formulated to be consumed or administered enterally under the supervision of a physician and which is intended for the specific dietary management of a disease or condition for which distinctive nutritional requirements, based on recognized scientific principles, are established by medical evaluation.”

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

Many embodiments of the invention are suitable for topical administration to a subject. Non-limiting examples of such embodiments comprise solutions, lotions, creams, ointments, gels, pastes, sprays, liquids, washes, hydrating agents or solutions, and perfusing agents or solutions. Topical doses of a compositions is higher than those doses if administered orally or intravenously, for example, as getting across the skin often requires a higher dose. Such doses can comprise those that are 2 times, 3 times, 4 times, 5 times, 6 times, 7 times, 8 times, 9 times, 10 times, or greater than 10 times the oral dose. Such doses can comprise those that are 2 times, 3 times, 4 times, 5 times, 6 times, 7 times, 8 times, 9 times, 10 times, or greater than 10 times the intravenous dose.

Compositions as described herein can comprise a synergistic combination of at least one flavanone and at least one flavonol, optionally comprising at least one carotenoid. The term “synergistic” can refer to the efficacy of the combination being more effective than the additive effects of either single therapy alone. The term “combination” can refer to a fixed combination in one dosage unit form, or a kit of parts for the combined administration where a compound and a combination partner (e.g., another drug, also referred to as “therapeutic agent” or “co-agent”) can be administered independently at the same time or separately within time intervals, especially where these time intervals allow that the combination partners show a cooperative, e.g., synergistic effect. The terms “co-administration” or “combined administration” or the like as utilized herein can encompass administration of the selected combination partner to a single subject in need thereof (e.g., a patient), and can include treatment regimens in which the agents are not necessarily administered by the same route of administration or at the same time.

The term “pharmaceutical combination” can refer to a product that results from the mixing or combining of more than one active ingredient and includes both fixed and non-fixed combinations of the active ingredients.

The term “fixed combination” can refer to active ingredients, e.g., a compound and a combination partner, that are both administered to a patient simultaneously in the form of a single entity or dosage. The term “non-fixed combination” can refer to the active ingredients, e.g., a compound and a combination partner, that are both administered to a patient as separate entities simultaneously, concurrently or sequentially with no specific time limits, wherein such administration provides therapeutically effective levels of the two compounds in the body of the patient. The latter also applies to cocktail therapy, e.g., the administration of three or more active ingredients.

The term “simultaneously” can refer to actions occurring at the same time or at about the same time, such as within about one minute of each other or within about five minutes of each other.

“Concurrently” can refer to actions occurring sufficiently close in time to produce a combined effect (e.g., concurrently can be simultaneously, or it can be two or more events occurring within a time period before or after each other).

The term “sequentially” can refer to actions in a sequence or a series of events. For example, the term can refer to administration of one active agent after another, e.g., within about 60 minutes of each other.

Embodiments can be administered alone to a subject, in combination with another pharmaceutical drug, as part of treatment regimen, or a component of a kit. In some embodiments, the other pharmaceutical drug is a drug used to a metabolic disorder or contribute to localized fat reduction. For example, such metabolic conditions can be diabetes, metabolic syndrome, or obesity, and such other pharmaceutical drug can be insulin, amylinomimetic agents, alpha-glucosidase inhibitors, biguanides, dopamine agonists, glucagon-like peptides, meglitinides, sodium glucose transporter 2 inhibitors, sulfonylureas, thiazolidinediones, and dipeptidyl peptidase-4 inhibitors. In some embodiments, the drug comprises regular insulin such as but not limited to Humulin or Novolin, insulin aspart such as but not limited to Novolog or FlexPen; insulin glulisine such as but not limited to Apidra; insulin lispro such as but not limited to Humalog; insulin isophane such as but not limited to Humulin N or Novolin N; insulin degludec such as but not limited to Tresiba; insulin detemir such as but not limited to Levemir; insulin glargine such as but not limited to Lantus; insulin glargine such as but not limited to Toujeo; a combination insulin drug such as but not limited to insulin aspart protamine-insulin aspart, insulin lispro protamine-insulin lispro, human isophane insulin-human insulin regular, insulin dedludec-insulin aspart, NovoLog Mix 70/30, Humalog Mix 75/25, Humalog Mix 50/50, Humalin 70/30, Novolin 70/30, or Ryzodeg; pramlintide such as but not limited to SymlinPen; acarbose such as but not limited to Precose; miglitol such as but not limited to Glyset; metformin such as but not limited to Glucophage, Metformin Hydrochloride ER, Glumetza, Riomet, or Fortamet; a metformin-containing drug such as but not limited to metformin-alogliptin, Kazano, metformin-canagliflozin, Invokamet, metformin-dapagliflozin, Xigduo XR, metformin-empagliflozin, Synjardy, metformin-glipizide, metformin-glyburide, Glucovance, metformin-linagliptin, Jentadueto, metformin-pioglitazone, Actoplus, Actoplus Met, Actoplus Met XR, metformin-repaglinide, PrandiMet, metformin-rosiglitazone, Avandamet, metformin-saxagliptin, Kombiglyze XR, metformin-sitagliptin, Janumet, or Janumet XR; bromocriptine such as but not limited to Parlodel; alogliptin such as but not limited to Nesina; alogliptin-pioglitazone such as but not limited to Oseni; linagliptin such as but not limited to Tradjenta, linagliptin-empagliflozin such as but not limited to Glyzami; saxagliptin such as but not limited to Onglyza; sitagliptin such as but not limited to Januvia; sitagliptin and simvastatin such as but not limited to Juvisync; albiglutide such as but not limited to Tanzeum; dulaglutide such as but not limited to Trulicity; exenatide such as but not limited to Byetta; exenatide extended-release such as but not limited to Bydureon; liraglutide such as but not limited to Victoza; nateglinide such as but not limited to Starlix; repaglinide such as but not limited to Prandin; dapagliflozin such as but not limited to Farxiga; canaglifoxin such as but not limited to Invokana; empaglifozin such as but not limited to Jardiance; empagliflozin-linagliptin such as but not limited to Glyxambi; glimepiride such as but not limited to Amaryl; glimepiride-pioglitazone such as but not limited to Duetact; glimepiride-rosiglitazone such as but not limited to Avandaryl; gliclazide, glipizide such as but not limited to Glucotrol; glyburide such as but not limited to DiaBeta, Glynase, or Micronase; chlorpropamide such as but not limited to Diabinese; tolazamide such as but not limited to Tolinase; tolbutamide such as but not limited to Orinase or TolTab; rosiglitazone such as but not limited to Avandia; or pioglitazone such as but not limited to Actos. In some embodiments, the treatment regimen includes administration of one or more pharmaceutical drugs, each administered separately to a subject; behavioral modification such as dietary changes and increased daily exercise; or surgery such as bariatric surgery. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The compositions of the disclosure can be formulated as a unit dosage form. In such form, the preparation is subdivided into unit doses containing appropriate quantities of the active ingredients such as for a single administration. The unit dosage form can be a packaged preparation, the package containing discrete quantities of preparation, for example, an ampule, a dispenser, an adhesive bandage, a non-adhesive bandage, a wipe, a baby wipe, a gauze, a pad and a sanitary pad.

The quantity of active compound in a unit dose of preparation can be varied or adjusted according to the instant application.

Compositions of the disclosure can be presented in a pack or dispenser device, such as a kit, which can contain one or more unit dosage forms containing the active ingredient. The pack can, for example, comprise metal or plastic foil, such as a blister pack. The pack or dispenser device can be accompanied by instructions for administration. The pack or dispenser device can also be accompanied by a notice in a form prescribed by a governmental agency regulating the manufacture, use, or sale of pharmaceuticals, which notice is reflective of approval by the agency of the form of the compositions for human or veterinary administration. Such notice, for example, can include labeling approved by the U.S. Food and Drug Administration for prescription drugs or of an approved product insert. Compositions comprising a preparation of the invention formulated in a pharmaceutically acceptable carrier can also be prepared, placed in an appropriate container, and labeled for treatment of an indicated condition, as detailed herein.

Since the compositions of the disclosure are utilized in vivo, the compositions can be of high purity and substantially free of harmful contaminants, e.g., at least National Food (NF) grade. Compositions as described herein can be at least analytical grade. Compositions as described herein can be at least pharmaceutical grade. To the extent that a given compound must be synthesized prior to use, such synthesis or subsequent purification can result in a product that is substantially free of any contaminating toxic agents that can have been used during the synthesis or purification procedures.

Aspects of the disclosure are also drawn towards methods of treating a subject afflicted with a metabolic disorder comprising administering to the subject a therapeutically effective amount of a composition described herein.

The term “treating” can refer to partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as a metabolic disorder. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

Therapeutic effects of treatment include, without limitation, preventing occurrence or recurrence of disease, alleviation of symptoms, diminishment of any direct or indirect pathological consequences of the disease, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment can prevent the onset of the disorder or a symptom of the disorder in a subject at risk for the disorder or suspected of having the disorder.

The term “subject” or “patient” can refer to any organism to which aspects of the invention can be administered, e.g., for experimental, diagnostic, prophylactic, and/or therapeutic purposes. Subjects to which compounds described herein can be administered can be mammals, for example primates, (such as humans). For veterinary applications, a wide variety of subjects will be suitable, e.g., livestock such as cattle, sheep, goats, cows, swine, and the like; poultry such as chickens, ducks, geese, turkeys, and the like; and domesticated animals for example, pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted herein or another organism that is alive. The term “living subject” refers to the entire subject or organism and not just a part excised (e.g., a liver or other organ) from the living subject.

The term “metabolism” can refer to, for example, the sum of the processes by which a substance is handled in the living body, and/or the sum of the metabolic activities taking place. For example, metabolism can refer to the chemical changes in living cells by which energy is provided for vital processes and activities and new material is assimilated.

The term “metabolic health” can refer to the presence or absence of metabolic disease. For example, metabolic health can refer to having less than ideal or ideal levels of blood sugar, triglycerides, high-density lipoprotein (HDL) cholesterol, blood pressure, and/or waist circumference. For example, a subject with poor metabolic health can be afflicted with or at risk of a metabolic disease. Such subject can have less than ideal levels of blood sugar, triglycerides, high-density lipoprotein (HDL) cholesterol, blood pressure, and/or waist circumference.

The term “metabolic disorder” can refer to any disorder that involves an alteration in the normal metabolism of carbohydrates, lipids, proteins, nucleic acids, or a combination thereof. A metabolic disorder can be associated with a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., type 1 diabetes, type 2 diabetes, gestational diabetes), hyperglycemia, hyperinsulinemia, insulin resistance, metabolic syndrome, and obesity.

The term “metabolic syndrome” can refer to a cluster of metabolic abnormalities including abdominal obesity, insulin resistance, glucose intolerance, diabetes, hypertension and dyslipidemia. These abnormalities are known to be associated with an increased risk of vascular events.

The term “obesity” can refer to a condition in which there is an excess of body fat. The operational definition of obesity is based on the Body Mass Index (BMI), which is calculated as body weight per height in meters squared (kg/m²). “Obesity” can refer to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². As used herein, the term “obesity” is meant to encompass the definitions of obesity herein.

Obesity-induced or obesity-related co-morbidities include, but are not limited to, diabetes, non-insulin dependent diabetes mellitus-type 2, diabetes associated with obesity, impaired glucose tolerance, impaired fasting glucose, insulin resistance syndrome, dyslipidemia, hypertension, hypertension associated with obesity, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. Co-morbidities can include without limitation: hypertension, hyperlipidemia, dyslipidemia, glucose intolerance, cardiovascular disease, sleep apnea, diabetes mellitus, and other obesity-related conditions.

Treatment of obesity and obesity-related disorders can refer to the administration of the compounds or combinations described herein to reduce or maintain the body weight of an obese subject. One outcome of treatment can be reducing the body weight of an obese subject relative to that subject's body weight immediately before the administration of the compounds or combinations described herein. Another outcome of treatment can be preventing body weight regain of body weight previously lost as a result of diet, exercise, or pharmacotherapy. Another outcome of treatment can be decreasing the occurrence of and/or the severity of obesity-related diseases. The treatment can suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment can also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders can refer to the administration of the compounds or combinations described herein to reduce or maintain the body weight of a subject at risk of obesity. One outcome of prevention can be reducing the body weight of a subject at risk of obesity relative to that subject's body weight immediately before the administration of the compounds or combinations described herein. Another outcome of prevention can be preventing body weight regain of body weight previously lost as a result of diet, exercise, or pharmacotherapy. Another outcome of prevention can be preventing obesity from occurring if the treatment is administered prior to the onset of obesity in a subject at risk of obesity. Another outcome of prevention can be decreasing the occurrence and/or severity of obesity-related disorders if the treatment is administered prior to the onset of obesity in a subject at risk of obesity. Moreover, if treatment is commenced in already obese subjects, such treatment can prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

The term “diabetes,” as used herein, includes both insulin-dependent diabetes mellitus (i.e., IDDM, also known as type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type 2 diabetics are also obese. The compositions of the disclosure are useful for treating both Type 1 and Type 2 diabetes. The compositions are especially effective for treating Type 2 diabetes. The compositions described herein are also useful for treating and/or preventing gestational diabetes mellitus.

Treatment of diabetes mellitus can refer to the administration of a compound or combination of the invention to treat diabetes. One outcome of treatment can be decreasing the glucose level in a subject with elevated glucose levels. Another outcome of treatment can be decreasing insulin levels in a subject with elevated insulin levels. Another outcome of treatment is decreasing plasma triglycerides in a subject with elevated plasma triglycerides. Another outcome of treatment is decreasing LDL cholesterol in a subject with high LDL cholesterol levels. Another outcome of treatment is increasing HDL cholesterol in a subject with low HDL cholesterol levels. Another outcome of treatment is increasing insulin sensitivity. Another outcome of treatment can be enhancing glucose tolerance in a subject with glucose intolerance. Yet another outcome of treatment can be decreasing insulin resistance in a subject with increased insulin resistance or elevated levels of insulin.

Prevention of diabetes mellitus refers to the administration of a compound or combination described herein to prevent the onset of diabetes in a subject in need thereof.

According to the invention, the term “NASH” or “Non-Alcoholic SteatoHepatitis” refers to a Non-Alcoholic Fatty Liver Disease condition characterized by the concomitant presence of liver steatosis, hepatocyte ballooning and liver inflammation at histological examination, (i.e. NAS>3, with at least 1 point in steatosis, at least 1 point in lobular inflammation and at least 1 point in the hepatocyte ballooning scores) in the absence of excessive alcohol consumption and after excluding other liver diseases like viral hepatitis (HCV, HBV). Embodiments can prevent the progression of NASH, which includes, for example, spider hemangioma, ascites, splenomegaly, hardening of the liver's edge, palm erythema, flapping tremor, liver fibrosis, one or more symptoms of degeneration and hepatocellular carcinoma. Increased nonalcoholic steatohepatitis is also associated with symptoms such as cirrhosis and liver failure, and is associated with liver transplantation.

The term “administration” or “administering” can refer to the physical introduction of an agent (e.g., a therapeutically effective amount of compounds or compositions described herein) into a subject using a variety of methods and delivery systems known to those of ordinary skill in the art. Modes of administration include those disclosed herein. Exemplary routes of administration include, for example, intravenous, intramuscular, subcutaneous, intraperitoneal, spinal or other parenteral routes of administration by injection or infusion. The phrase “parenteral administration” as used herein can refer to modes of administration other than enteral and topical administration, generally by injection, and includes, but is not limited to, intravenous, intramuscular, intraarterial, intrathecal, intralymphatic, intralesional, intracystic, intraorbital, intracardiac, intradermal, intraperitoneal, cervical, subcutaneous, subepidermal, intraarticular, subcapsular, subarachnoid, intrathecal, epidural and intrasternal injections and infusions, as well as in vivo including electroporation. In some embodiments, the formulation is administered via a parenteral route, eg, orally. Other parenteral routes include topical, epidermal or mucosal routes of administration, eg, intranasally, vaginally, rectally, sublingually or topically. Administration can be performed, for example, once, several times, and/or over one or more extended periods of time.

Aspects of the disclosure are drawn towards methods of weight and fat reduction. The term “body fat” can refer to the loose connective tissue known as “adipose tissue”. Body fat can be present throughout the body of an individual. Non-limiting examples of the body fat (adipose tissue) include visceral fat, perirenal fat, mesenteric fat, epididymal fat, and subcutaneous fat. The amount of body fat in an individual can be determined and/or estimated by a variety of methods identifiable to a skilled person. For example, body fat percentage (mass of body fat divided by body mass) can be estimated by techniques known to a skilled person such as hydrostatic (underwater) weighing, whole-body air displacement plethysmography, near-infrared interactance, DEXA (Dual Energy X-ray Absorptiometry), body average density measurement (in conjunction with use of the Brozek or Siri formulas), bioelectrical impedance analysis, anthropometric methods (e.g. skinfold measurements, ultrasound measurements, and estimations based on the subject's body mass index), magnetic resonance imaging, computed tomography, and other methods identifiable to a skilled person. Additionally, though not a direct measurement of body fat amount, an individual's body mass index (BMI) can also be indicative of the amount of body fat in an individual. Additional methods for determining and/or estimating the amount of body fat will be identifiable to a skilled person.

The term “reducing” as in “reducing body fat” as used herein refers to a lowering in the amount, mass, or volume of body fat. For example, “weight and body fat reduction” can refer to the presence of a reduced amount of weight or body fat after administration of a therapeutically effective amount of compounds or compositions described herein. The term “reduce” can refer to diminishing the volume, size, mass, bulk, density, amount, and/or quantity of a substance. Such reduction can be measured and determined by measuring the amount of fat according to one or more of the methods described herein at an initial time point prior to the administering of the compounds or compositions described herein and then measuring the amount of body fat at various time points (e.g. during the period of administering the compounds described herein as well after the administering has ceased). For example, a subject's body weight can be measured prior to beginning a treatment regimen with the compounds described herein and then measured during and after the treatment regimen. A decrease in body weight is indicative of a reduction in body fat. Similarly, skinfold measurements and/or other techniques (e.g. magnetic resonance imaging and/or computerized tomography) can be made or performed along with the weight measurements where a decrease in the parameters measured by those techniques (i.e. body fat percentage) is indicative of fat reduction. Additionally, the reduction of fat can be determined qualitatively such as by photographing the whole body, or portions of the body, at various time points before, during, and after a treatment regimen where the reduction in fat can be determined by visual inspection of the images (e.g. by seeing a visible reduction and in the size and/or volume of a fat deposit such as submental fat, waist fat, cellulite, and other forms of body fat amenable to visual inspection). The methods of the invention described herein can reduce body fat by about 5%, by about 10%, or by about 20% or more of the total weight of the individual. For example, this translates into a weight loss of about 2 to 3 pounds per week for an individual. In one embodiment, the amount of weight loss can be about 1% body fat/week.

Since other modifications and changes varied to fit operating requirements and environments will be apparent to those skilled in the art, the invention is not considered limited to the example chosen for purposes of disclosure, and covers all changes and modifications which do not constitute departures from the true spirit and scope of this invention. This written description provides an illustrative explanation and/or account of the present invention. It can be possible to deliver equivalent benefits using variations of the specific embodiments, without departing from the inventive concept. This description and these drawings, therefore, are to be regarded as illustrative and not restrictive.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the properties sought to be obtained by the present disclosure. At the very least, and not as an attempt to limit the application of the doctrine of equivalents to the scope of the claims, each numerical parameter can at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” can be used to refer to one or more specified characteristics of a plurality of previously recited elements or steps. When used with the open-ended term “comprising,” the recitation of the term “each” does not exclude additional, unrecited elements or steps. Thus, it will be understood that an apparatus can have additional, unrecited elements and a method can have additional, unrecited steps, where the additional, unrecited elements or steps do not have the one or more specified characteristics.

Aspects of the invention are directed towards kits. The term “kit” can refer to a set of articles that facilitates the process, method, assay, analysis, or manipulation of a sample. The kit can include instructions for using the kit (eg, instructions for the method of the invention), materials, solutions, components, reagents, chemicals, or enzymes required for the method, and other optional components.

For example, the compositions as described herein can be provided in a kit. In one embodiment, the kit includes (a) a container that contains the compositions described herein or components thereof (e.g., solvents, buffers, extracts, botanical compounds), and optionally (b) informational material. The informational material can be descriptive, instructional, marketing or other material that encompasses the methods described herein and/or the use of the agents for therapeutic benefit. In an embodiment, the kit also includes a second agent, such as at least one additional active agent.

The informational material of the kits is not limited in its form. In one embodiment, the informational material can include information about production of the compound, molecular weight of the compound, concentration, date of expiration, batch or production site information, and so forth. In one embodiment, the informational material encompasses methods of manufacturing one or more compositions, and/or methods of administering compositions to a subject, e.g., in a suitable dose, dosage form, or mode of administration (e.g., a dose, dosage form, or mode of administration described herein), to treat a subject. The information can be provided in a variety of formats, include printed text, computer readable material, video recording, or audio recording, or information that provides a link or address to substantive material.

The composition in the kit can include other ingredients, such as a solvent or buffer, culture media, a stabilizer, or a preservative. The compositions of the kit thereof can be provided in any form, e.g., liquid, dried or lyophilized form, and can be substantially pure and/or sterile. When the compositions are provided in a liquid solution, the liquid solution can be an aqueous solution or an alcohol solution. When the compositions or components thereof are provided as a dried form, reconstitution, for example, is by the addition of a suitable solvent. The solvent, e.g., sterile water or buffer, can optionally be provided in the kit. The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes the composition and a second agent, e.g., in a desired ratio. For example, the kit includes a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Synergy of Naringenin and Myricetin to Increase UCP-1 and GLUT-4

We previously found that naringenin, an extract of sweet oranges, increased UCP-1 and GLUT-4 in human fat cells and tested 150 mg of naringenin three times a day in a patient with type 2 diabetes and found an increase in insulin sensitivity, metabolic rate and weight loss. We subsequently found that beta carotene was synergistic with naringenin in increasing UCP-1 and GLUT-4 in human fat cells. We demonstrated that larger doses of naringenin are safe in a pharmacokinetic study, and a study is about to start testing the synergistic dose of naringenin and beta carotene in a person with diabetes evaluating metabolic rate, insulin resistance and body weight.

We have now demonstrated in human fat cells that myricetin is synergistic with naringenin giving many fold greater increase in UCP-1 and GLUT-4. This invention of synergy of myricetin and naringenin to increase UCP-1 and GLUT-4 can represent a more effective and safe treatment for obesity and type 2 diabetes. Obesity and overweight now affects over half of the adult United States Population and an increase in the prevalence of type 2 diabetes follows the prevalence of obesity by about a decade. Not only is the increasing obesity pandemic fueling an increase in type 2 diabetes, but a treatment for type 2 diabetes that causes weight loss is useful in combating these two health problems and improving public health. Since both naringenin and myricetin are foods, they are safe, inexpensive and will only require efficacy trials to be sold without a prescription. Thus, without wishing to be bound by theory, naringenin and myricetin can provide a cost-effective treatment for both problems due to the lack of safety studies needed for new chemical entities and have a big public health impact.

Pharmaceuticals are very expensive, primarily due to the fact that most are new chemical entities that have strong patents, but have never been used in humans. Thus the FDA has safety studies that are needed to insure safety before approval for use to treat disease. These studies to establish safety are very expensive and the cost of pharmaceuticals is steadily taking a bigger share of our healthcare expenses. Myricetin and naringenin are both foods and are Generally Recognized As Safe (GRAS). Efficacy trials giving statistically and clinically significant weight loss and improvement in type 2 diabetes are relatively inexpensive. Thus, not only are the myricetin and naringenin inexpensive food, they also do not have the development costs of pharmaceuticals. Thus, without wishing to be bound by theory, they can provide a safer and effective treatment for obesity and type 2 diabetes at a much lower cost. A better product at a lower cost has the potential to be a big economic success.

### Example 2

### Myricetin for Treatment of Metabolic Diseases with Naringenin

Myricetin is a flavonoid that can be found in many common foods such as berries, vegetables, fruits, tea and red wine. It is extracted from all parts of the Chinese bayberry tree Myrica Rubra in Japan where it is used to flavor snack foods and drinks. It is listed as GRAS (generally recognized as safe) by the Flavor and Extract Manufacturer Association and was recently judged to be safe at current estimated dietary exposure by the Joint Food and Agriculture Organization of the United Nations (FAO)/World Health Organization (WHO) Expert Committee on Food Additives (JECFA). Specifications related to the identity and purity of myricetin for use as a flavoring agent have been established by JECFA [1]. Without wishing to be bound by theory, studies on cells in vitro and in rodents indicates that myricetin can be a therapeutic for many diseases including diabetes, cancer, cardiovascular disease, and Alzheimer's disease [2].

Chemical properties: Myricetin has six hydroxyl groups and is aqueous soluble at 17 μg/ml (11 nM). The solubility increases in acetic acid buffered solutions as pH is reduced. The solubility in an acidic solution with pH equal to 1, 2 or 3 is 777 μg/ml, 576 μg/ml and 150 μg/ml respectively. In rats myricetin is absorbed in the duodenum because of its weak acidity, and it has moderate membrane permeability with a coefficient of 1.7×10−6 cm/s [3]. Myricetin is stable at 25° C. with a half-life of 23 hours.

The oral bioavailability of myricetin is approximately 10% and is dose-dependent in rats orally administered 50 mg/kg and 100 mg/kg with a time to maximal concentration (Tmax) of 6.5 hours [4]. It reportedly inhibits cytochrome P450 and drug efflux pumps like P-glycoprotein to enhance availability of co-administered drugs such as tamoxifen [5] and losartan in rats [6].

Effects on biological pathways: Myricetin lowers inflammation in cell culture models by reducing TNF-α, and inhibiting several pathways including Toll receptor activity, NF-kB and ikB, Jak/STAT1 and MAPK [2]. It also reduces oxidative stress by increasing cellular activity of the antioxidant enzymes superoxide dismutase, glutathione peroxidase, catalase and GSH in rodent models and macrophage cultures. The inhibitory effects of myricetin on human cancer cell invasion and proliferation have been documented in numerous studies using lung, breast, liver, ovarian and colon cancer cells[7]. Myricetin reduces MTORC1 and MTORC2 activity in glioblastoma cells, reduces VEGF and neovascularization, promotes apoptosis by downregulating Bcl-2, reduces invasion and metastasis by lowering expression of matrix metalloproteases, and inhibits the STAT3 pathway[7].

Obesity, diabetes and fatty liver disease: Studies in rodents indicate that myricetin can treat obesity and diabetes by altering expression of genes regulating energy expenditure. Myricetin increases mitochondrial density in muscle and BAT, muscle endurance capacity, and BAT energy expenditure genes by elevating SIRT1 activity[8, 9]. In mice fed a high fat and sucrose diet, the cohort with 0.12% myricetin included in the diet had reductions in adiposity, and circulating glucose and insulin[10]. In mice, myricetin induces mitochondrial oxygen consumption rate and improved glucose tolerance by upregulation of Sirt3 in adipose tissue[11]. Myricetin has activity as a GLP-1R agonist at low micromolar concentrations. In rat islets incubated in high glucose myricetin stimulated insulin release, and a single dose injection in rats maintained reduced blood glucose levels for eight hours[12]. Oral administration of myricetin in rats on a high fat diet improved fatty liver disease and lowered circulating triglycerides[13].

PPARγ is a key transcriptional activator of genes that promote insulin sensitivity and adipogenesis. Cyclin-dependent kinase 5 (CDK5) can phosphorylate PPARγ on a specific serine residue and this modification regulates PPARγ target gene selectivity. Inhibition of CDK5 in adipose tissue is sufficient to stimulate the anti-diabetic effects of PPARγ while blocking adipogenic effects that cause weight gain[14]. Multiple flavonols including myricetin, quercetin and kaemferol inhibit CDK5 kinase activity with an EC50 in the low micromolar range [15]. CDK5 is expressed in pancreatic B-cells, and myricetin treatment of β-cells exposed to high glucose prevented apoptosis and potentiated insulin secretion through multiple mechanisms including CDK5 inhibition [16].

Multiple clinical trials indicate that supplements containing myricetin improve type 2 diabetes. Blueberin, a supplement composed of blueberry leaves with 50 mg myricetin and 50 mg chlorogenic acid, was administered three times a day in a 4-week randomized placebo-controlled clinical trial. Blueberin significantly reduced fasting plasma glucose from 143+/−5.2 mg/L to 104+/−5.7 mg/L (p<0.001) compared to placebo and had anti-inflammatory effects[17]. Emulin, a supplement containing myricetin, chlorogenic acid and quercetin was evaluated with metformin in a randomized, double-blind, placebo controlled, parallel group study on individuals with type 2 diabetes and BMI≥30. Emulin increased the efficacy of metformin 30-fold for improving fasting glucose, whereas Emulin alone and metformin alone were not statistically different [18]. A Chinese population study showed that higher levels of dietary myricetin correlated with a lower prevalence of type 2 diabetes[19].

### Characterization of Naringenin Effects in Human Adipocytes

Our research group has demonstrated that naringenin and naringenin extract (30% from whole Citrus sinensis) stimulate expression of genes for fat oxidation, energy expenditure and insulin sensitivity in human adipose tissue[20]. Naringenin also upregulates expression of secreted factors that increase whole body energy expenditure including adiponectin, ANGPTL4, GDF11 and PM20D1. Naringenin activates PPARα and PPARγ (Goldwasser), and studies with selective inhibitors indicate that activity of PPARα and PPARγ are required for the effects of naringenin on gene expression in human adipocytes[21]. In a case report, an individual with obesity and diabetes lost weight, had improved insulin levels and a measurable increase in energy expenditure after ingesting 150 mg naringenin in an-extract-of sweet oranges (Citrus sinensis) three times a day for eight weeks

### Flavonols Enhance the Effects of Naringenin on Gene Expression in Human Adipocytes

Quercetin, kaempferol and myricetin inhibit CDK5 with EC50s of 63, 66 and 71, respectively[15]. In adipocytes, CDK5 inhibitors reduce adipogenesis and increase genes for energy expenditure by altering PPARγ target gene selection. We measured the expression of the PPARα/γ direct target gene UCP1 after treating human adipocyte cultures with CDK5-inhibiting flavonols combined with naringenin for seven days. We estimated the human Cmax for myricetin to be approximately 2.5 μM based on rodent pharmacokinetic studies[4], and all flavonols were evaluated at that concentration. GLUT4 expression, an indicator of insulin sensitivity that is upregulated after conversion of white adipocytes to beige adipocytes, was also evaluated.

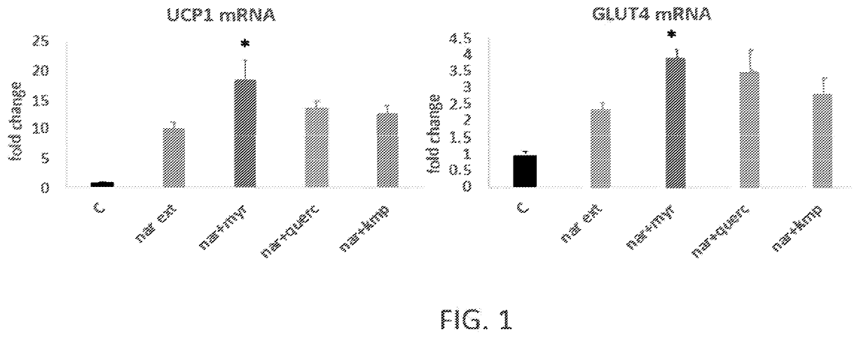

Myricetin was more effective in upregulating gene expression than quercetin or kaempferol when combined with naringenin (FIG. 1). Myricetin was the only flavonol that synergistically increased expression of UCP1 and GLUT4 compared to naringenin alone, although it is a weaker CDK5 inhibitor than quercetin or kaempferol.

Myricetin Combined with Naringenin has Therapeutic Effects on Obesity and Diabetes

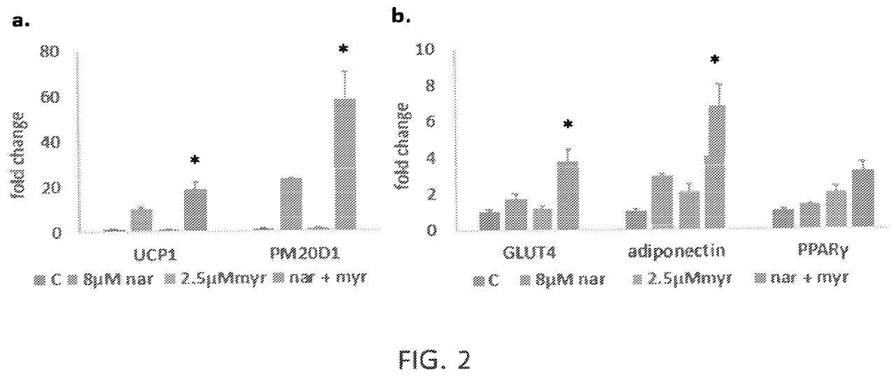

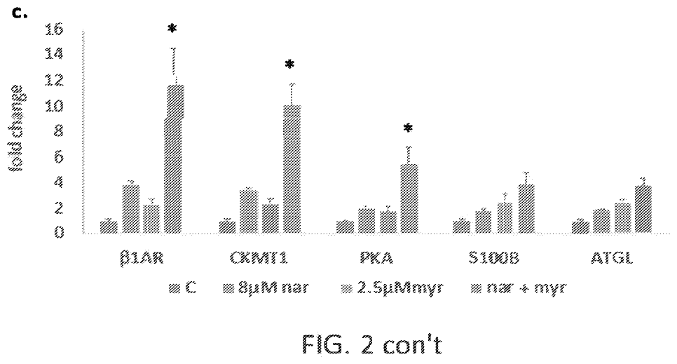

Treatment of adipocytes with the combination of myricetin and naringenin synergistically increased key genes for human lipolysis and energy expenditure including UCP1, peptidase M20 domain containing 1 (PM20D1)[23], mitochondrial creatine kinase 1 (CKMT1)[24], β1 adrenergic receptor (β1AR)[25, 26], and protein kinase A regulatory subunit 2B (PKA)[27] (FIG. 2). The glucose transporter GLUT4, and adiponectin which is a secreted protein that increases whole-body insulin sensitivity, were also synergistically upregulated. PPARγ, neurotrophic factor S100B[28] and adipose triglyceride lipase (ATGL)[29] increased but were not synergistic with naringenin.

Based on the strong effects on gene expression shown above, without wishing to be bound by theory, myricetin and naringenin combined can have greater efficacy for the treatment of obesity and diabetes compared to naringenin alone[22].

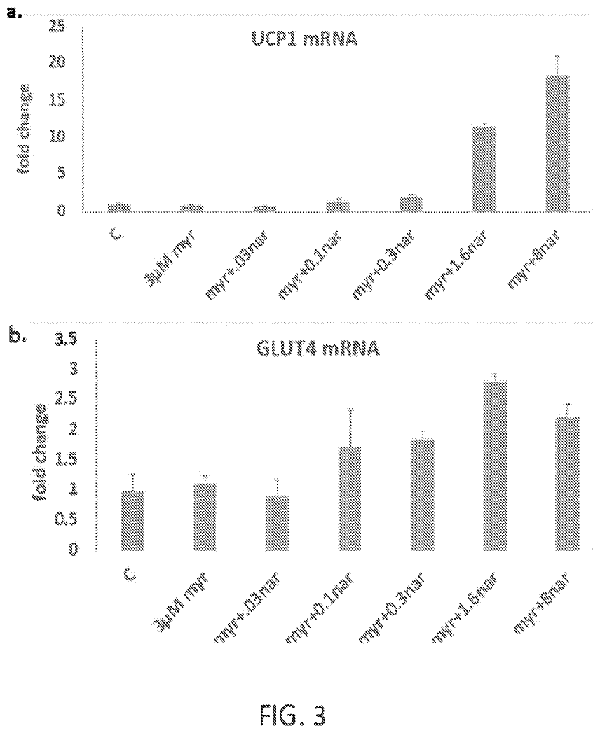

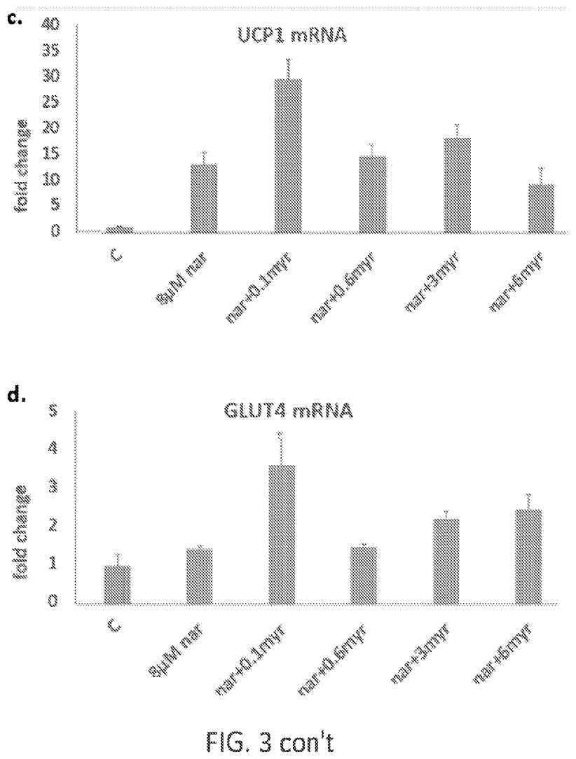

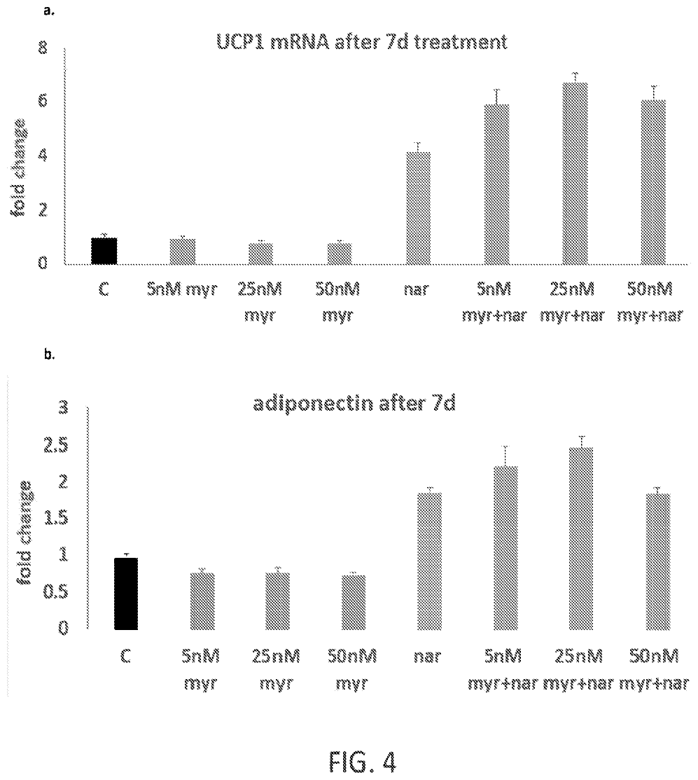

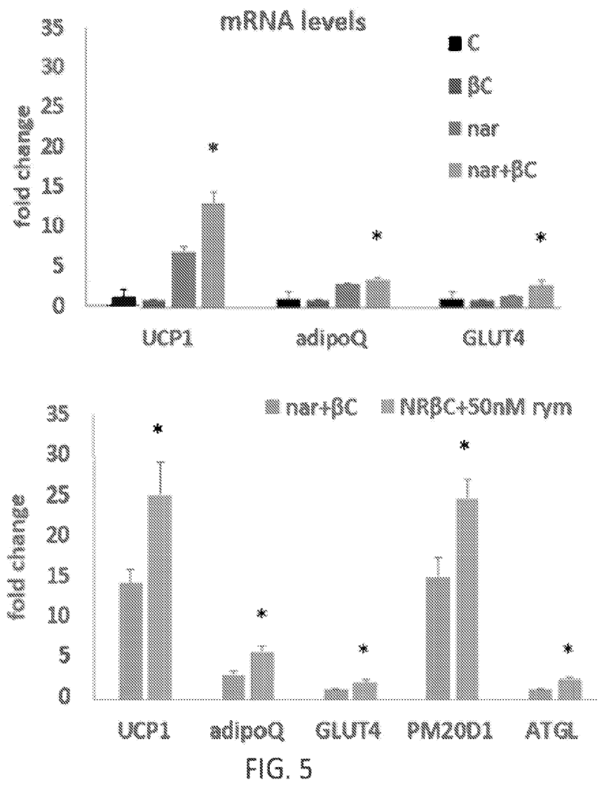

The upregulation of gene expression by myricetin after addition to 8 μM naringenin was most potent at 0.1 μM, the lowest concentration of myricetin tested in this assay. The effect decreased at higher concentrations (FIG. 3, panels c and d). There are no published human pharmacokinetic studies of the myricetin Cmax or Tmax. These results indicate that myricetin can be effective even at much lower concentrations than our estimated human Cmax.

### Myricetin Enhances the Effects of Naringenin on Gene Expression at Low Nanomolar Concentrations

To determine the lowest concentration of with activity, myricetin was tested at low nanomolar concentrations. Treatment for 7 days with myricetin alone had no effect at any concentration. Combined with naringenin the effects of myricetin remained strong at 50 nM and 25 nM, and were slightly reduced at 5 nM.

The Combination of Myricetin with Naringenin and β-Carotene has the Highest Potency to Induce Expression of Genes for Thermogenic Energy Expenditure, Insulin Sensitivity and Lipolysis

β-carotene (βC), a vitamin A precursor and ligand for the PPARγ coactivator RXR, synergistically enhances the effects of naringenin on gene expression in human adipocytes (Provisional Patent submitted). The addition of nanomolar concentrations of myricetin further upregulates gene expression.

### Summary

Naringenin, a flavonoid found in sweet oranges, activates PPARα and PPARγ in human adipocytes and induces expression of genes for thermogenesis and insulin sensitivity. We conducted a pharmacokinetic study and demonstrated its safety and bioavailability at doses ranging from 150 mg to 900 mg[30]. In a case study the lowest dose of 150 mg was administered for 8 weeks to a subject with obesity and diabetes and resulted in the reduction of body weight and fasting blood insulin concentration, and an increase in metabolic rate.

Addition of beta-carotene to naringenin during treatment of human fat cells synergistically increases expression of genes for thermogenic energy expenditure and insulin sensitivity.

In the current application, we show that addition of myricetin to naringenin alone or to the combination of naringenin and beta carotene approximately doubles the upregulation of thermogenic gene expression in human adipocytes, as well as the secreted insulin sensitizing hormone adiponectin. Myricetin, naringenin and beta-carotene will make a potent therapeutic treatment for metabolic diseases including obesity, NASH and diabetes.

### REFERENCES CITED IN THIS EXAMPLE

1. Hobbs, C. A., et al., Genotoxicity evaluation of the flavonoid, myricitrin, and its aglycone, myricetin. Food Chem Toxicol, 2015. 83: p. 283-92.
2. Song, X., et al., Myricetin: A review of the most recent research. Biomed Pharmacother, 2021. 134: p. 111017.
3. Yao, Y., et al., Preformulation studies of myricetin: a natural antioxidant flavonoid. Pharmazie, 2014. 69(1): p. 19-26.
4. Dang, Y., et al., Quantitative determination of myricetin in rat plasma by ultra performance liquid chromatography tandem mass spectrometry and its absolute bioavailability. Drug Res (Stuttg), 2014. 64(10): p. 516-22.
5. Li, C., et al., Effects of myricetin, an anticancer compound, on the bioavailability and pharmacokinetics of tamoxifen and its main metabolite, 4-hydroxytamoxifen, in rats. Eur J Drug Metab Pharmacokinet, 2011. 36(3): p. 175-82.
6. Choi, D. H., C. Li, and J. S. Choi, Effects of myricetin, an antioxidant, on the pharmacokinetics of losartan and its active metabolite, EXP-3174, in rats: possible role of cytochrome P450 3A4, cytochrome P450 2C9 and P-glycoprotein inhibition by myricetin. J Pharm Pharmacol, 2010. 62(7): p. 908-14.
7. Jiang, M., et al., Anti-tumor effects and associated molecular mechanisms of myricetin. Biomed Pharmacother, 2019. 120: p. 109506.
8. Hu, T., et al., Myricetin-induced brown adipose tissue activation prevents obesity and insulin resistance in db/db mice. Eur J Nutr, 2018. 57(1): p. 391-403.
9. Jung, H. Y., et al., Myricetin improves endurance capacity and mitochondrial density by activating SIRT1 and PGC-1 alpha. Scientific Reports, 2017. 7.
10. Choi, H. N., et al., Ameliorative effect of myricetin on insulin resistance in mice fed a high-fat, high-sucrose diet. Nutrition Research and Practice, 2014. 8(5): p. 544-549.
11. Akindehin, S., et al., Myricetin Exerts Anti-Obesity Effects through Upregulation of SIRT3 in Adipose Tissue. Nutrients, 2018. 10(12).
12. Li, Y., et al., Myricetin: a potent approach for the treatment of type 2 diabetes as a natural class B GPCR agonist. Faseb Journal, 2017. 31(6): p. 2603-2611.
13. Sun, W. L., et al., Myricetin supplementation decreases hepatic lipid synthesis and inflammation by modulating gut microbiota. Cell Rep, 2021. 36(9): p. 109641.
14. Banks, A. S., et al., An ERK/Cdk5 axis controls the diabetogenic actions of PPAR gamma. Nature, 2015. 517(7534): p. 391-U581.
15. Zapata-Torres, G., et al., Effects of natural flavones and flavonols on the kinase activity of Cdk5. Journal of Natural Products, 2004. 67(3): p. 416-420.
16. Karunakaran, U., et al., Myricetin Protects Against High Glucose-Induced beta-Cell Apoptosis by Attenuating Endoplasmic Reticulum Stress via Inactivation of Cyclin-Dependent Kinase 5. Diabetes & Metabolism Journal, 2019. 43(2): p. 192-205.
17. Abidov, M., et al., Effect of Blueberin on fasting glucose, C-reactive protein and plasma aminotransferases, in female volunteers with diabetes type 2: double-blind, placebo controlled clinical study. Georgian Med News, 2006(141): p. 66-72.
18. Ahrens, M. J. and D. L. Thompson, Effect of emulin on blood glucose in type 2 diabetics. J Med Food, 2013. 16(3): p. 211-5.
19. Yao, Z., et al., Dietary myricetin intake is inversely associated with the prevalence of type 2 diabetes mellitus in a Chinese population. Nutr Res, 2019. 68: p. 82-91.
20. Rebello, C. J., et al., Naringenin Promotes Thermogenic Gene Expression in Human White Adipose Tissue. Obesity (Silver Spring), 2019. 27(1): p. 103-111.
21. Goldwasser, J., et al., Transcriptional regulation of human and rat hepatic lipid metabolism by the grapefruit flavonoid naringenin: role of PPARalpha, PPARgamma and LXRalpha. PLOS One, 2010. 5(8): p. e12399.
22. Murugesan, N., et al., Naringenin Increases Insulin Sensitivity and Metabolic Rate: A Case Study. J Med Food, 2020. 23(3): p. 343-348.
23 Long, J. Z., et al., The Secreted Enzyme PM20D1 Regulates Lipidated Amino Acid Uncouplers of Mitochondria. Cell, 2016. 166(2): p. 424-435.
24. Muller, S., et al., Proteomic Analysis of Human Brown Adipose Tissue Reveals Utilization of Coupled and Uncoupled Energy Expenditure Pathways. Sci Rep, 2016. 6: p. 30030.
25 Riis-Vestergaard, M. J., et al., Beta-1 and Not Beta-3 Adrenergic Receptors May Be the Primary Regulator of Human Brown Adipocyte Metabolism. J Clin Endocrinol Metab, 2020. 105(4).
26. Mattsson, C. L., et al., beta (1)-Adrenergic receptors increase UCP1 in human MADS brown adipocytes and rescue cold-acclimated beta (3)-adrenergic receptor-knockout mice via nonshivering thermogenesis. Am J Physiol Endocrinol Metab, 2011. 301(6): p. E1108-18.
27. Mantovani, G., et al., Protein kinase A regulatory subunits in human adipose tissue: decreased R2B expression and activity in adipocytes from obese subjects. Diabetes, 2009. 58(3): p. 620-6.
28. Zeng, X., et al., Innervation of thermogenic adipose tissue via a calsyntenin 3beta-S100b axis. Nature, 2019. 569(7755): p. 229-235.
29 Khan, S. A., et al., ATGL-catalyzed lipolysis regulates SIRT1 to control PGC-1alpha/PPAR-alpha signaling. Diabetes, 2015. 64(2): p. 418-26.
30. Rebello, C. J., et al., Safety and pharmacokinetics of naringenin: A randomized, controlled, single-ascending-dose clinical trial. Diabetes Obes Metab, 2020. 22(1): p. 91-98.

### Example 3

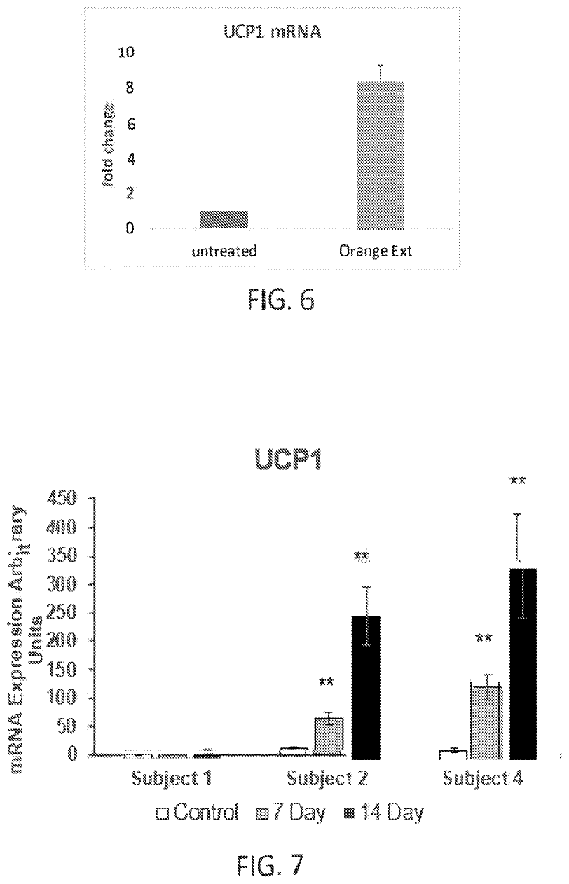

Referring to FIG. 6 and FIG. 7, naringenin stimulates metabolism in human fat. We were able to show using human fat cells in culture and adult adipose specimens removed during surgeries that naringenin caused an increase in Uncoupling Protein-1 (UCP-1). UCP-1 is an indication that the fat cells are generating energy as heat which is another way of saying that the fat cells are increasing their metabolic rate.

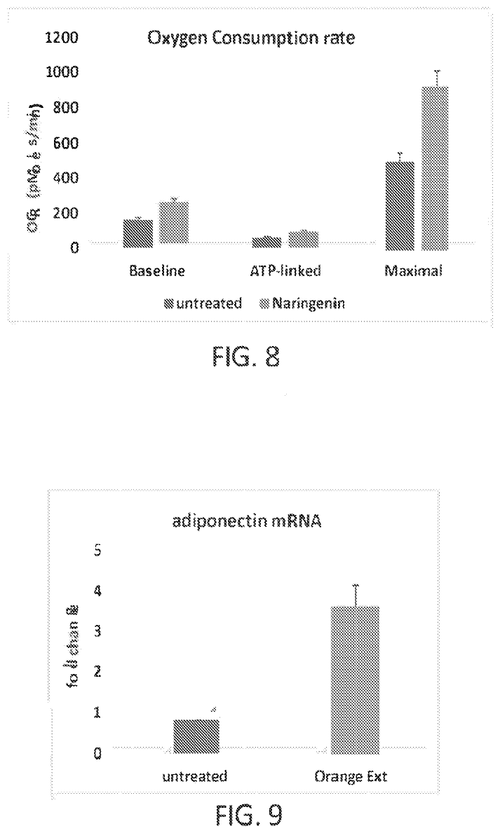

Referring to FIG. 8, cells consume oxygen while metabolizing fat for thermogenic energy expenditure, and we directly measured oxygen consumption rate. Naringenin is a strong inducer of basal and maximal energy expenditure in human fat cells.

Referring to FIG. 9, naringenin promotes insulin sensitivity. Naringenin also increased production of a secreted hormone (adiponectin) which increases uptake of glucose by many tissues and reduces insulin resistance which is the underlying cause of type 2 diabetes.

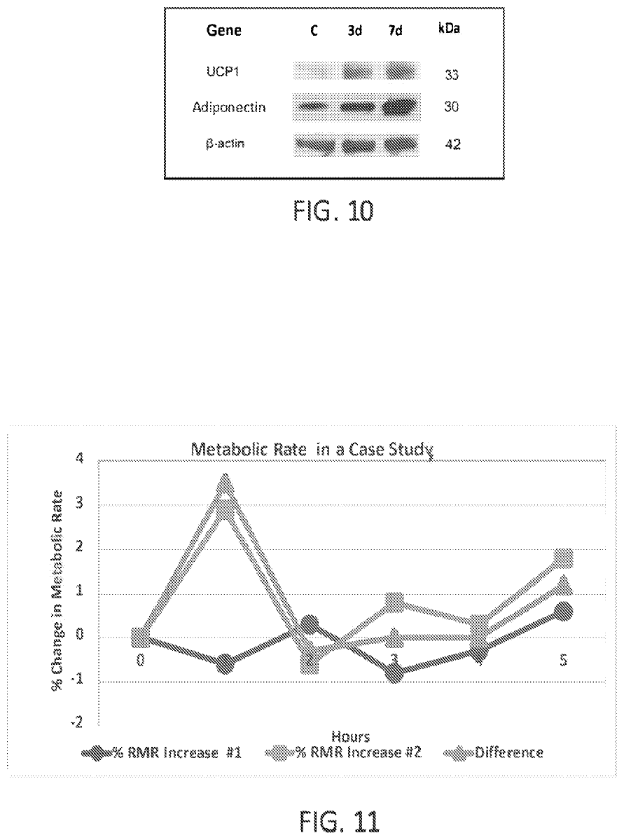

Referring to FIG. 10, naringenin stimulates increases in protein levels. In addition to upregulating messenger RNA, protein levels for UCP-1 and adiponectin increased. Beta-actin is the loading control.

Referring to FIG. 11, naringenin shows efficacy at a low dose in a human. Next, we gave 150 mg of naringenin extract in 500 mg of orange extract three times a day by capsule to a woman who was obese and had type 2 diabetes that was only treated with diet. She took the capsule three times a day for 8 weeks without going on a diet or changing her exercise habits. She lost 2.3% of her body weight which indicates that when her weight loss reached a plateau at about 6 months, her weight loss can be 4.6% of her body weight. For an obesity drug to get approval for weight loss it should cause a loss of 5%, so the results were close to what one can expect for a drug. Her insulin resistance went down by 20% and her metabolic rate went up by 3 percent.

We have subsequently conducted a pharmacokinetic study showing that doses of naringenin up to 900 mg were safe and without side effects. The pharmacokinetic study demonstrated that doses of up to 900 mg were safe and without side effects. We went back to the woman whom we tested at 150 mg three times a day with naringenin, and tested her with 300 mg three times a day. She maintained the loss on the lower dose of naringenin and had also lost further weight, so she was below her natural weight set point. The 300 mg three times a day only maintained her weight loss without adding further weight loss. The weight loss she achieved after the testing of the 150 mg dose had resolved her diabetes.

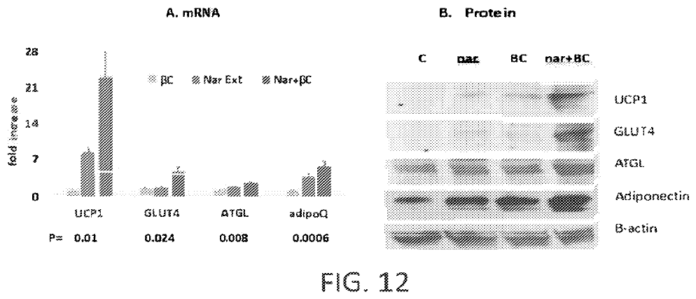

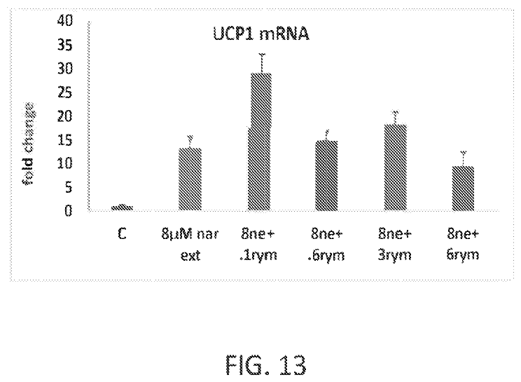

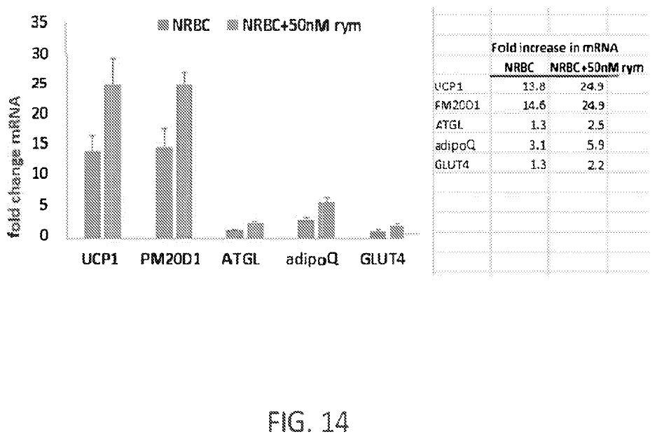

Referring to FIG. 12, the addition of β-carotene (BC) to 8 μM naringenin extract synergistically induces gene expression in human adipocytes treated for 7 days. We showed that both mRNA and protein levels of genes for energy expenditure and insulin sensitivity increased significantly with the combination compared to naringenin extract alone.

Testing naringenin extract 300 mg three times a day with beta carotene 7.5 mg twice a day in humans. We are now treating a series of humans with obesity who need to lose weight to undergo bariatric surgery safely. These human subjects will be treated for 8 weeks and have body weight, fasting insulin and fasting glucose measured at baseline and 8 weeks. Without wishing to be bound by theory, a loss of body weight and an improvement in insulin sensitivity.

Naringenin extract combined with β-carotene and rym (myricetin). The flavonoid myricetin (rym) regulates biological pathways activated by naringenin at a different level than β-carotene. We examined the effects of rym at different concentrations and found potent upregulation of naringenin activity in human adipocytes. We observed that rym enhances the activity of naringenin at low concentrations.

The addition of myricetin (rym) to nar ext+β-carotene (NRBC) almost doubles the effects on gene expression. We tested the ability of rym to enhance the potency of the NRBC combination. Rym activity was evaluated in combination with NRBC at 0.1 μM (100 nM) and at lower concentrations of 50 nM and 25 nM.

These effects were observed for both energy expenditure genes and insulin sensitivity genes, indicating that a supplement containing all three compounds can be a potent therapeutic for weight loss and insulin sensitivity. Because rym has activity at low concentrations, approximately 15 mg per capsule can be sufficient to achieve blood concentrations in this range.

Combining naringenin 300 mg and rym 15 mg 3 times/day with beta carotene 7.5 mg twice/day. Without wishing to be bound by theory, the effects of adding naringenin to rym and beta carotene will give the greatest weight loss and improvement in insulin sensitivity. A case series of obese humans who need to lose weight prior to bariatric surgery will have baseline and 8-week measures of body weight, fasting insulin and fasting glucose.

We will validate the triple combination in a double-blind clinical trial with measures of body weight, metabolic rate measured over 4 hours, fasting insulin, and fasting glucose at baseline and at the end of the study which can extend 8, 12, 16 or 24 weeks.

### Example 4

### Background of Patented Discoveries in the Human Obesity Laboratory

Our goal is to develop a safe, potent combination of natural compounds targeting energy expenditure and the insulin sensitivity in adipose tissue for treatment of obesity and diabetes. Most currently approved obesity drugs target the brain to reduce appetite and can have off-target side effects.

### Naringenin

Adipose tissue is a complex tissue composed of multiple types of adipocytes which vary in function. White adipocytes store triglycerides and expand in number and size under conditions of excess energy intake. White adipocytes can be converted into beige adipocytes with a high level of mitochondrial energy expenditure. A marker of beige adipocytes is expression of mitochondrial uncoupling protein 1 (Ucp1), a protein that facilitates thermogenesis and fatty acid metabolism. Beige adipocytes secrete factors into circulation that increase whole body fat and glucose metabolism, insulin sensitivity and reduce appetite.

Our lab discovered that treatment of human white fat cells with 8 μm naringenin (NR), a flavonoid from oranges, can turn on expression of “beige” genes for thermogenesis and insulin sensitivity. We also showed that naringenin requires both transcriptional activators PPARγ and PPARα to reprogram gene expression.

Next, we conducted clinical studies to determine whether naringenin works as a therapeutic for weight loss and diabetes. First, a single dose randomized pharmacokinetic analysis showed that a dose of 150 mg NR has a Cmax of approximately 16 μM and the half-life is 3 hours. Circulating levels reach 48 μm at a 600 mg dose and there are no adverse events at a dose of 900 mg. In a case study of an individual with obesity and untreated diabetes, ingestion of 150 mg of NR three times a day for eight weeks reduced body weight by 2.3%, reduced fasting insulin concentrations by 20%, and produced a 3.5% increase in energy expenditure. These clinical data indicate that naringenin is bioavailable and has potential as a therapeutic for obesity and diabetes. For these reasons, we consider adipose tissue a relevant peripheral target for obesity drugs.

### Naringenin and β-Carotene

More recently we discovered that the effects of NR on expression of thermogenic genes were amplified by adding 2 μm β-carotene (BC) to NR during treatment. BC is a food component that is converted into retinoic acid, a ligand for the retinoic X receptor (RXR). PPARγ and PPARα each can form heterodimers with RXRs to regulate gene expression. In human adipocytes, NR combined with BC (NRBC) amplified UCP1 expression and genes for several other energy-dissipating pathways, beneficial secreted peptides and adipokines.

### Naringenin, β-Carotene and Myricetin

We discovered that myricetin approximately doubles the effects of NR+BC by upregulating beige genes for thermogenesis and insulin sensitivity. Myricetin is most active at nanomolar concentrations (50 to 100 nM).

### Example 5

Naringenin (NR) acts on human adipocytes by binding the nuclear receptors PPARg and PPARα to regulate target genes for thermogenesis and insulin sensitivity. Addition of myricetin (myr) to NR amplifies expression of a number of these target genes, including UCP1, GLUT4, PM20D1, adiponectin, PPARg, B1AR, CKMT1, ATGL, S100b and PKA, compared to NR alone in adipocytes. We investigated several mechanisms for the effects of myricetin on gene expression based on published literature including upregulation of SIRT1 and SIRT3 expression by myricetin, induction of PGC-1α protein, and inhibition of CDK5 phosphorylation of PPARg at serine 273. In some experiments we have analyzed the combination of all three compounds.

Myricetin does not Enhance SIRT1 or SIRT3 Expression Compared to NR Alone

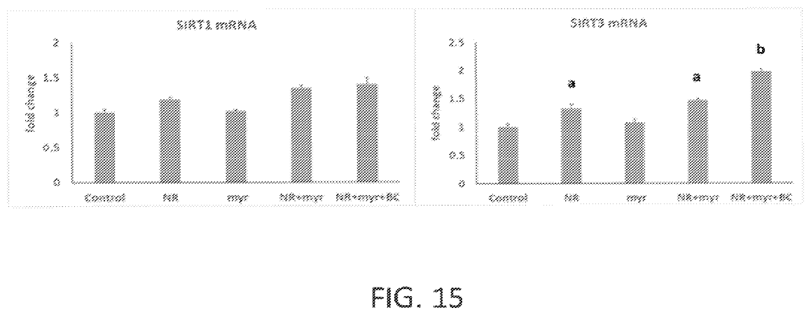

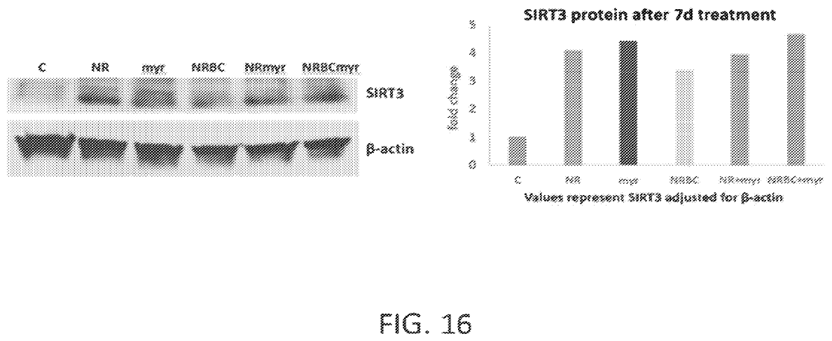

Myricetin increases levels of multiple Sirt proteins in adipocytes from mice, including SIRT1, Sirt3 and SIRT5. This study indicated that Sirt3 was the specific protein responsible for the anti-obesity effects of myricetin in mice fed a high fat diet¹. Sirt proteins have deacetylase activity and can act on transcriptional factors such as PPARg to upregulate target gene expression. Sirt1 and Sirt3 are known to act on PPARg in adipocytes, so we analyzed whether mRNA levels were affected by myricetin in our model of human adipocytes. Human adipocytes were treated for seven days with 8 μM NR and 50 nM myricetin, and the results are shown in FIG. 15. There was no effect of myr treatment alone on SIRT1 or SIRT3 mRNA levels. Myricetin+NR did not increase expression of SIRT1 or SIRT3 compared to NR alone (FIG. 15). Addition of 2 μM BC to the NR+myr combination significantly increased SIRT3 expression in adipocytes compared to NR or NR+myricetin.

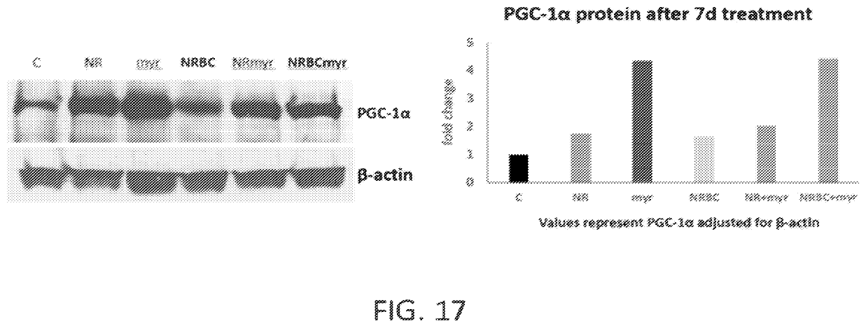

Since we saw an increase in SIRT3 mRNA and myricetin upregulates SIRT3 protein in adipocytes from mice, we analyzed levels of SIRT3 protein in human adipocytes treated for seven days with the same concentrations of compounds. Protein was isolated from adipocytes and analyzed on immunoblots probed with antibodies that specifically bind human SIRT3 or human β-actin. FIG. 16 shows a Western blot of SIRT3 protein from human adipocytes. The histogram on the right shows values of SIRT3 protein normalized for the loading control β-actin. The results shown in FIG. 16 indicate that NR and myricetin each upregulate SIRT3 protein levels, but the combination does not further increase levels. Combining NR and myr did not increase SIRT3 protein compared to NR or myr alone. However, adding myr to NR+BC boosted SIRT3 protein by 37%.

Elevated levels of SIRT3 can result in deacetylation and upregulation of PPARg and PPARα activity. PGC-1α is another coactivator of both PPARs, and it is known to be a target of SIRT3 deacetylation and activation². It is possible that adding myricetin can boost gene expression higher than NR+BC by elevating SIRT3 and the subsequent deacetylation of PPARg, PPARα and PGC-1α.

### Myricetin Induces PGC-1α Protein Levels

PGC-1α is an important coactivator of both PPARg and PPARα for mitochondrial genes involved in thermogenesis and energy expenditure³. Myricetin increases Pgc-1α a in metabolic tissues of mice^{1,4}. Elevated PGC-1α protein drives increases in target genes for PPARg and PPARα^{5,6}. We evaluated PGC-1α protein levels in human adipocytes treated for seven days with 8 μM NR, 2 μM BC and 50 nM myricetin.

We found that myricetin robustly upregulated levels of PGC-1α protein, shown in FIG. 17. NR induced levels approximately two-fold compared to the untreated control, and the NRBC combination also increased protein two-fold. Myricetin addition to NR induced a small increase in PGC-1α. Myricetin addition to NRBC upregulated PGC-1α levels over two-fold compared to NRBC. The increase in PGC-1a protein provides that adding myricetin to NRBC can elevate mitochondrial biogenesis as well as other target genes.

### Myricetin Did not Reduce PPARg Phosphorylation at Ser-273 Compared to NRBC

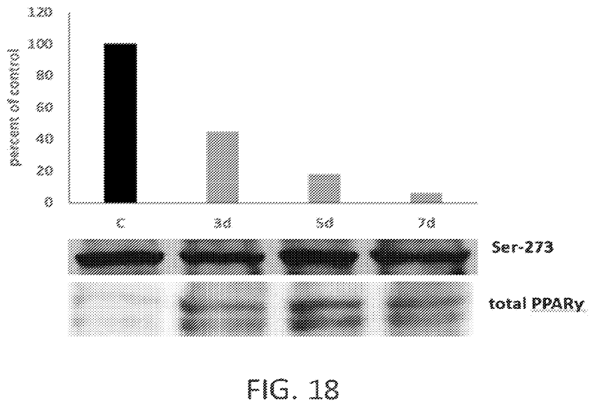

Phosphorylation of PPARg by CDK5 at serine-273 is an important mechanism for inhibition of target genes for insulin sensitivity⁷. Inhibitors of CDK5 have potential as diabetes drugs⁸. Several studies have provided that myricetin is a CDK5 inhibitor and has insulin sensitizing characteristics that make it a potential therapeutic for diabetes^{9,10}. We previously analyzed NRBC for effects on PPARg phosphorylation at serine 273. Human adipocytes were treated with 8 μM NR and 2 μM BC for three, five and seven days, and protein was isolated and analyzed on immunoblots probed with an antibody specific for human P-serine 273 PPARg. The same blot was stripped and re-probed with an antibody for total human PPARg. Proteins were quantified and results are shown in FIG. 18. Values in the histogram represent (P-ser 273 PPARg)/(total PPARg). Treatment of human adipocytes with NRBC induces PPARg protein levels and concurrently inhibits P-serine 273 phosphorylation. The effect increases over time.

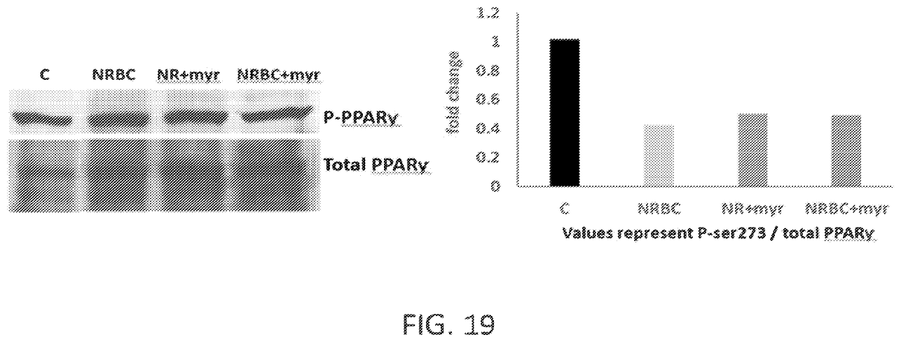

The experiment for analysis of phosphorylation of PPARg at P-273 was repeated to determine whether adding myricetin to NRBC can further inhibit PPARg P-273 phosphorylation. Cells were treated for seven days with 8 μM NR, 2 μM BC and 50 nM myricetin. Protein was analyzed on immunoblots probed with the same antibodies. The data in FIG. 19 shows that addition of myricetin to NRBC did not further decrease PPARg P-273 relative to NRBC.

### Summary

Myricetin addition to NR did not increase SIRT3 protein levels. Addition of myr to the combination of NRBC increased SIRT3 mRNA and protein two-fold. Addition of myricetin to NRBC amplified PGC-1α protein levels two-fold higher than NR alone or NRBC. Myricetin did not alter phosphorylation of PPARg at serine-273.

### REFERENCES CITED IN THIS EXAMPLE

1. Akindehin, S., Jung, Y. S., Kim, S. N., Son, Y. H., Lee, I., Seong, J. K., Jeong, H. W., and Lee, Y. H. (2018). Myricetin Exerts Anti-Obesity Effects through Upregulation of SIRT3 in Adipose Tissue. Nutrients 10. 10.3390/nu10121962.
2. Shi, T., Wang, F., Stieren, E., and Tong, Q. (2005). SIRT3, a mitochondrial sirtuin deacetylase, regulates mitochondrial function and thermogenesis in brown adipocytes. J Biol Chem 280, 13560-13567. 10.1074/jbc.M414670200.
3. Puigserver, P., Wu, Z., Park, C. W., Graves, R., Wright, M., and Spiegelman, B. M. (1998). A cold-inducible coactivator of nuclear receptors linked to adaptive thermogenesis. Cell 92, 829-839. 10.1016/s0092-8674(00)81410-5.
4. Jung, H. Y., Lee, D., Ryu, H. G., Choi, B. H., Go, Y., Lee, N., Lee, D., Son, H. G., Jeon, J., Kim, S. H., et al. (2017). Myricetin improves endurance capacity and mitochondrial density by activating SIRT1 and PGC-1 alpha. Sci Rep-Uk 7. ARTN 6237
10.1038/s41598-017-05303-2.
5. Puigserver, P., and Spiegelman, B. M. (2003). Peroxisome proliferator-activated receptor-gamma coactivator 1 alpha (PGC-1 alpha): transcriptional coactivator and metabolic regulator. Endocr Rev 24, 78-90. 10.1210/er.2002-0012.
6. Mazzucotelli, A., Viguerie, N., Tiraby, C., Annicotte, J. S., Mairal, A., Klimcakova, E., Lepin, E., Delmar, P., Dejean, S., Tavernier, G., et al. (2007). The transcriptional coactivator peroxisome proliferator activated receptor (PPAR) gamma coactivator-1 alpha and the nuclear receptor PPAR alpha control the expression of glycerol kinase and metabolism genes independently of PPAR gamma activation in human white adipocytes. Diabetes 56, 2467-2475. 10.2337/db06-1465.
7. Choi, J. H., Banks, A. S., Estall, J. L., Kajimura, S., Bostrom, P., Laznik, D., Ruas, J. L., Chalmers, M. J., Kamenecka, T. M., Bluher, M., et al. (2010). Anti-diabetic drugs inhibit obesity-linked phosphorylation of PPARgamma by Cdk5. Nature 466, 451-456. 10.1038/nature09291.
8. Choi, J. H., Banks, A. S., Kamenecka, T. M., Busby, S. A., Chalmers, M. J., Kumar, N., Kuruvilla, D. S., Shin, Y., He, Y., Bruning, J. B., et al. (2011). Antidiabetic actions of a non-agonist PPARgamma ligand blocking Cdk5-mediated phosphorylation. Nature 477, 477-481. 10.1038/nature10383.
9. Karunakaran, U., Elumalai, S., Moon, J. S., Jeon, J. H., Kim, N. D., Park, K. G., Won, K. C., Leem, J., and Lee, I. K. (2019). Myricetin Protects Against High Glucose-Induced beta-Cell Apoptosis by Attenuating Endoplasmic Reticulum Stress via Inactivation of Cyclin-Dependent Kinase 5. Diabetes Metab J 43, 192-205. 10.4093/dmj.2018.0052.
10. Zapata-Torres, G., Opazo, F., Salgado, C., Munoz, J. P., Krautwurst, H., Mascayano, C., Sepulveda-Boza, S., Maccioni, R. B., and Cassels, B. K. (2004). Effects of natural flavones and flavonols on the kinase activity of Cdk5. J Nat Prod 67, 416-420. 10.1021/np034011s.

### Example 6

### Pharmacokinetics of Myricetin

Myricetin supplement: 10 mg dose can give approximately 400 nM Cmax

Human Cmax: a 564 mg dose=39.4 μM Cmax (based on Table 1)

Conversion from rats:

50 mg/kg in rats is in humans 50/6.2 mg/kg (from rat myricetin table below)×70 kg=564 mg dose for human

A human has about 45 L of fluid, so 560 mg/45L=12.5 mg/L=12.5 ug/ul

$$If mw = 318 ( myricetin ) , 1 mM = \text{.318} mg / ml ( ug / ul ) so 12.5 / \text{.318} = 39.4 uM C max x = 10 \% bioavailability = 4 uM or 8 uM at 100 mg dose$$

Absolute bioavailability was found to be 9.62% and 9.74% at 2 oral doses (50 mg/kg and 100 mg/kg)

(Cayman 10012600) Cmax 39.4 μM, 10 μM used in cell culture, up to 100 μm used on primary islets for 24 h with no loss of viability (Karunakaran) dissolve in DMSO 5 mM=1.6 mg/ml, dilute 1:2000

From Table 1 (rat): 1.5 ug/ul=5 μM and 2.6 ug/ul=8.7 μM Cmax

TABLE 1
Conversion Table showing Parameters and Routes of Administration for Myricetin.

| | Administration routes | | |
|---|---|---|---|
| Parameters | p.o. | | i.v. |
| Dose (mg/kg) | 50 | 100 | 0.5 |
| Cₘₐₓ (ng/mL)^{a} | 1488.75 ± 200.78 | 2611.76 ± 1019.58 | 2232.16 ± 856.36 |
| Tₘₐₓ (h)^{b} | 6.4 ± 0.89 | 5.2 ± 3.03 | 0 (pre-dose) |
| t_{1/2} (h) | 2.78 ± 0.81 | 3.79 ± 0.75 | 6.99 ± 1.94 |
| AUC_{0-∞}(ng · h/mL)^{c} | 13,731.13 ± 5,574.58 | 28,081.62 ± 10,982.45 | 1452.85 ± 170.30 |
| CL (L/h/kg)^{d} | — | — | 0.35 ± 0.05 |
| V_{d} (L/kg)^{e} | — | — | 4.12 ± 1.52 |
| Bioavailability (%)^{f} | 9.62 | 9.74 | — |

^{a}The peak serum concentration of a drug after administration
^{b}The amount of time for which a drug is present at maximum concentration in serum
^{c}The total drug exposure over time
^{d}The rate at which the drug is cleared from the blood
^{e}Volume of distribution, a proportionality constant between total amount of drug in the body and plasma concentrations
^{f}Fraction of drug that reaches the blood stream when compared with the amount of the drug that administered

Tox: 1000 mg/kg/day mice=11 g for human

### REFERENCES CITED IN THIS EXAMPLE

Choi, Jang Hyun, et al. “Antidiabetic actions of a non-agonist PPARγ ligand blocking Cdk5-mediated phosphorylation.” Nature 477.7365 (2011): 477-481.
Akindehin, Seun, et al. “Myricetin exerts anti-obesity effects through upregulation of SIRT3 in adipose tissue.” Nutrients 10.12 (2018): 1962.
Dang, Y., et al. “Quantitative determination of myricetin in rat plasma by ultra performance liquid chromatography tandem mass spectrometry and its absolute bioavailability.” Drug research 64.10 (2014): 516-522.
Guo, Rui Xue, et al. “Preparation and characterization of microemulsions of myricetin for improving its antiproliferative and antioxidative activities and oral bioavailability.” Journal of agricultural and food chemistry 64.32 (2016): 6286-6294.
Hu, Tao, et al. “Myricetin-induced brown adipose tissue activation prevents obesity and insulin resistance in db/db mice.” European journal of nutrition 57 (2018): 391-403.
Karunakaran, Udayakumar, et al. “Myricetin protects against high glucose-induced β-cell apoptosis by attenuating endoplasmic reticulum stress via inactivation of cyclin-dependent kinase 5.” Diabetes & Metabolism Journal 43.2 (2019): 192-205.
Qian, Jin, et al. “Self-nanoemulsifying drug delivery systems of myricetin: formulation development, characterization, and in vitro and in vivo evaluation.” Colloids and Surfaces B: Biointerfaces 160 (2017): 101-109.

Zhu, Sifeng, et al. “Development of M10, myricetin-3-O-β-d-lactose sodium salt, a derivative of myricetin as a potent agent of anti-chronic colonic inflammation.” European Journal of Medicinal Chemistry 174 (2019): 9-15.

### Example 7

### Naringenin and β-Carotene Convert Human White Adipocytes to a Beige Phenotype and Elevate Hormone-Stimulated Lipolysis

### Abstract

Naringenin, a peroxisome proliferator-activated receptor (PPAR) activator found in citrus fruits, upregulates markers of thermogenesis and insulin sensitivity in human adipose tissue. Our pharmacokinetics clinical trial demonstrated that naringenin is safe and bioavailable, and our case report showed that naringenin causes weight loss and improves insulin sensitivity. PPARs form heterodimers with retinoic-X-receptors (RXRs) at promoter elements of target genes. Retinoic acid is an RXR ligand metabolized from dietary carotenoids. The carotenoid β-carotene reduces adiposity and insulin resistance in clinical trials. Our goal was to examine if carotenoids strengthen the beneficial effects of naringenin on human adipocyte metabolism. Human preadipocytes from donors with obesity were differentiated in culture and treated with 8 μM naringenin+2 μM β-carotene (NRBC) for seven days. Candidate genes involved in lipolysis, thermogenesis, and glucose metabolism were measured using qRT-PCR and immunoblotting. We found that carotenoids act synergistically with naringenin to boost UCP1 and glucose metabolism genes including GLUT4 and adiponectin, compared to naringenin alone. Protein levels of PPARα, PPARγ and PPARγ-coactivator-1α, key modulators of thermogenesis and insulin sensitivity, were also upregulated after treatment with NRBC. Transcriptome sequencing was conducted and the bioinformatics analyses of the data revealed that NRBC induced enzymes for several non-UCP1 pathways for energy expenditure including triglyceride cycling, creatine kinases, and Peptidase M20 Domain Containing 1 (PM20D1). A comprehensive analysis of changes in receptor expression showed that NRBC upregulated eight receptors that have been linked to lipolysis or thermogenesis including the β1-adrenergic receptor and the parathyroid hormone receptor. Lipolysis fuels thermogenesis, and NRBC increased levels of triglyceride lipases and agonist-stimulated lipolysis in adipocytes. We observed that expression of RXRγ, an isoform of unknown function, was induced ten-fold after treatment with NRBC. We show that RXRγ is a coactivator bound to the immunoprecipitated PPARγ protein complex from white and beige human adipocytes. In conclusion, NRBC upregulates the capacity for lipolysis and expression of key enzymatic pathways for thermogenesis in human adipocytes and has therapeutic potential.

### Introduction

Adipose tissue is a complex, adaptable organ composed of multiple types of adipocytes which vary in function (1). White adipocytes store triglycerides and expand in number and size under conditions of excess energy intake. Brown and beige adipocytes abound in mitochondria and express uncoupling protein 1 (UCP1), a protein that shifts mitochondrial fat oxidation away from ATP production and towards thermogenesis. Increased density of beige adipocytes and UCP1 in fat depots is associated with elevated energy expenditure and resistance to weight gain and type 2 diabetes (2, 3, 4). In rodents, white adipose tissues can adapt to a chronic environmental stimulus such as cold exposure by producing beige adipocytes from precursor cells and by converting white adipocytes into beige cells (5). The adaptive response to cold exposure involves release of norepinephrine by sympathetic nerve fibers to activate β3-adrenergic receptors (β3AR) abundantly expressed in adipocytes(6).

In contrast to rodents, human adipocytes express extremely low levels of the βARs. Humans lack a robust response to systemic infusion of β-adrenergic agonists even when combined with cold exposure (7, 8, 9). However, synthetic peroxisome proliferator activator receptor (PPAR)α and γ ligands have been shown to have potent activity in the conversion of primary human white adipocytes to beige UCP1-expressing cells in vitro (10, 11). PPARs are ligand-activated nuclear receptors enriched in metabolic tissues, and they regulate UCP1 and many genes controlling fat oxidation and insulin sensitivity by binding to upstream PPAR responsive elements (PPREs). PPARγ is subject to complex cell-specific post-translational regulatory mechanisms and has multiple ligand binding domains (12). Depending on the binding characteristics of a ligand, PPARγ can stimulate adipogenesis, insulin sensitivity, fat oxidation or thermogenesis in adipose tissues (13).

Thiazolidinediones (TZDs) are a class of potent PPARγ agonists that have been approved by the United States Food and Drug Administration (FDA) for the treatment of type 2 diabetes. However, TZDs have multiple adverse effects including weight gain, heart failure, and risk for bladder cancer (14, 15). PPARα agonists primarily upregulate genes for lipolysis and mitochondrial β-oxidation of fatty acids in primary human adipocytes(16). A synthetic PPARα activator, fenofibrate, has been approved for treatment of dyslipidemia (17, 18). At this time, there are no FDA-approved PPARγ or PPARα activators for treatment of obesity (19, 20, 21).

Despite the complex regulation of ligand binding to PPARγ, evidence is growing that selective modulators and partial agonists can direct activity toward thermogenesis and away from adipogenesis (12, 22, 23). Naringenin (NR), a polyphenol found in citrus fruit, activates transcriptional activity of PPARγ and PPARα in an expression system with a PPRE linked to a reporter gene (24). In a previous study, we treated human subcutaneous adipocytes with NR and saw induction of UCP1 mRNA as well as increases in basal and maximal oxygen consumption rate (OCR) (25). We used selective inhibitors to demonstrate that upregulation of thermogenesis genes by NR requires activation of both PPARγ and PPARα in adipocytes (26). Data from our clinical studies indicate that naringenin is bioavailable and has potential as a treatment for obesity and type 2 diabetes. Our pharmacokinetic clinical trial showed that naringenin is safe and well-tolerated at doses ranging from 150 mg to 900 mg (27). In a case study of an individual with obesity and untreated type 2 diabetes, we found that body weight and fasting insulin concentrations decreased and there was a measurable increase in energy expenditure after ingestion of NR for eight weeks (26).

Carotenoids are vitamin A precursors found in fruits and vegetables and their consumption has been shown to reduce fat mass and insulin resistance in children. Baseline blood concentrations of β-carotene (BC) are inversely correlated with fat mass (28, 29). Most carotenoids are metabolized into retinoid ligands for retinoic acid receptors (RAR) and retinoic-X-receptors (RXR), which are transcriptional coactivators for PPARs. The objective of this study was to determine whether carotenoids can amplify the effects of NR on gene expression and function to convert primary human adipocytes to a beige phenotype. We observed synergistic increases in a subset of mRNAs including UCP1, GLUT4, ATGL and adiponectin with the combination of NR and BC (NRBC). Protein levels of PPARα, PPARγ, PGC-1a and NAMPT were selectively upregulated without increases in mRNA levels. Whole transcriptome sequencing was conducted, and the results showed that NRBC induced genes for multiple non-UCP1 energy-dissipating futile cycles, beneficial secreted peptides, and adipokines. Importantly, NRBC increased levels of multiple thermogenesis and lipolysis-linked receptors including the β1-adrenergic receptor (β1AR), parathyroid hormone receptor (PTHR), and the stimulatory ratio of natriuretic peptide receptors (NPR1/NPR3). A comprehensive analysis of lipolysis was conducted, and the results showed that the capacity for PTHR and βAR agonist-stimulated lipolysis was substantially higher in NRBC-treated adipocytes compared to untreated cells. Expression of RXRγ, a unique isoform associated with brown adipogenesis, was upregulated ten-fold after treatment. We observed that RXRγ was bound in the PPARγ transcriptional complex immunoprecipitated from human adipocytes. These results indicate that NRBC has potential as a treatment for obesity and type 2 diabetes and is safe primarily because it acts on peripheral tissues, unlike most obesity medications that act on the central nervous system (CNS) (30).

### Results

Carotenoids Act Synergistically with NR to Amplify UCP1 Levels in Adipocytes

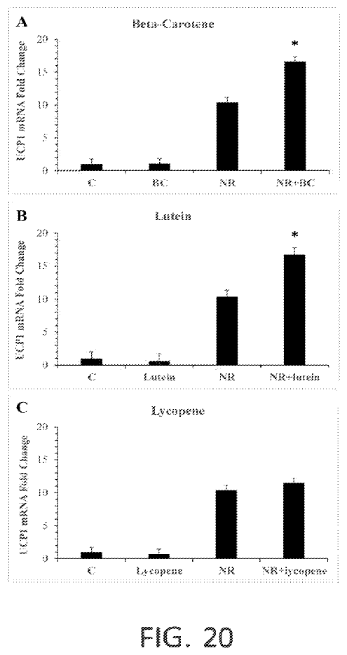

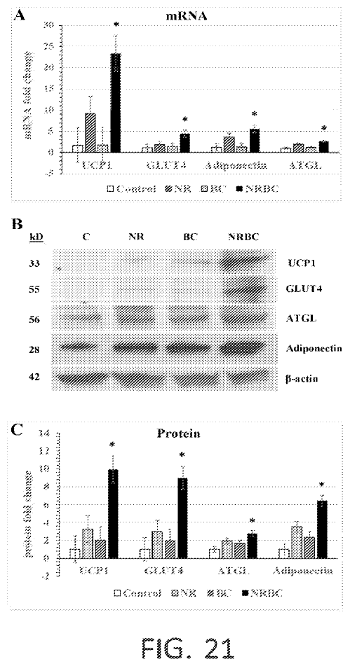

Metabolites of pro-vitamin A carotenoids are ligands of the retinoid X receptor (RXR) family of nuclear receptors which can heterodimerize with PPARα and PPARγ and recruit coactivators into active transcriptional complexes (46). Our first objective was to evaluate whether treatment of adipocytes with carotenoids can elevate the levels of UCP1, a PPAR target gene known to be upregulated by NR and a marker for beige adipogenesis (25). Three of the most abundant carotenoids in foods and in human serum, BC, lycopene and lutein were tested (47). Steady state plasma concentrations are approximately 2 μM in individuals who eat diets rich in carotenoids, and we used this concentration in experiments (48). Since the mean serum concentration of NR after ingestion of 150 mg is 8 μM (27), we used this concentration in all assays. Treatment of primary human adipocyte cultures with BC, lutein or lycopene individually for seven days had no effect on gene expression (FIG. 20). When BC or lutein were combined with NR for treatment, the increase in UCP1 gene expression was synergistic, greater than the sum of the individual effects of each compound (P<0.001). In contrast, lycopene did not alter NR-stimulated UCP1 levels. Unlike lycopene, BC and lutein are pro-vitamin A carotenoids that are metabolized into retinoic acid (49). These data show that RXR ligands act synergistically with NR to boost thermogenesis gene expression.

### BC Elevates the Effects of NR on a Subset of Genes for Thermogenesis and Insulin Sensitivity

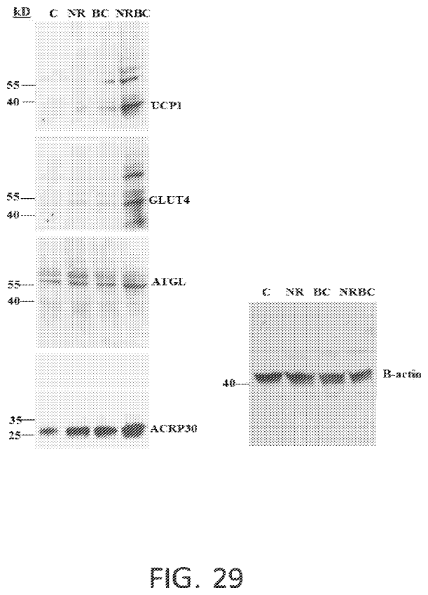

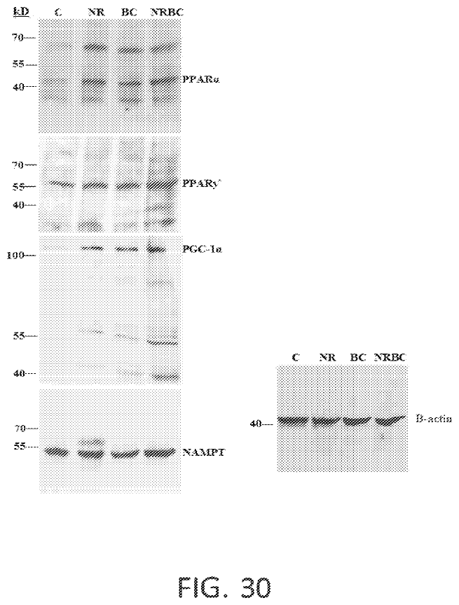

We next evaluated the effects of NR, BC and NRBC on expression of additional genes. BC was chosen for the rest of the study because it is safe, stable, bioavailable and has a long half-life in circulation (50). Adipocytes from five donors with body mass index (BMI) ranging from 27 to 36 kg/m² were treated with NRBC for seven days. NRBC synergistically boosted mRNA levels for UCP1 (p<0.005), GLUT4 (p<0.02), ATGL (p<0.04) and adiponectin (p<0.05) compared to NR or BC alone (FIG. 21a). Protein levels showed a similar trend (FIGS. 21b and c). Uncropped Western blots are shown in FIG. 29. ATGL is the rate-limiting lipase for hydrolysis of fatty acids from triglycerides (TGs) (51). GLUT4 is a transporter for glucose uptake, and its upregulation in adipocytes stimulates a cascade of events that reduce insulin resistance (52, 53). Adiponectin is a key circulating factor that acts on muscle and other tissues and improves whole body glucose homeostasis (54). In a previous study we reported induction of CPT1β mRNA in adipocytes after NR treatment (25). We did not see an increase in CPT1ß levels with NRBC treatment in comparison to levels induced by NR alone.

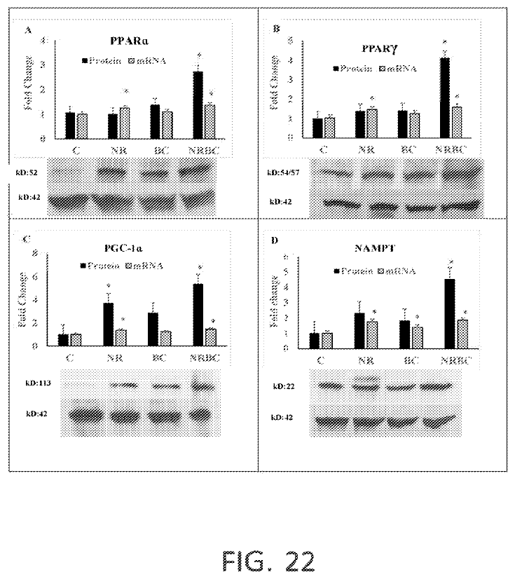

NRBC upregulated protein levels of PPARα, PPARγ, PGC-1α, and NAMPT three to six-fold (FIG. 22, p<0.001) without comparable increases in mRNA levels. Uncropped Western blots are shown in FIG. 30. PPARα, PPARγ, and PGC-1α proteins have a short half-life and are rapidly degraded by ubiquitin-proteosome systems (55, 56, 57). Inhibition of degradation increases protein levels and target gene expression (58). The binding of ligands and coactivators can influence the turnover rates of PPARα and PPARγ (59, 60). Treatment with NR alone did not upregulate protein levels, indicating that RXR ligand is required in addition to PPAR ligand for protein upregulation. The increase in these proteins in the absence of concurrent upregulation of their transcripts indicates that NR and BC act together to stabilize protein levels through post-translational mechanisms.

### Whole Transcriptome Sequencing for Comprehensive Analysis of Metabolic Effects

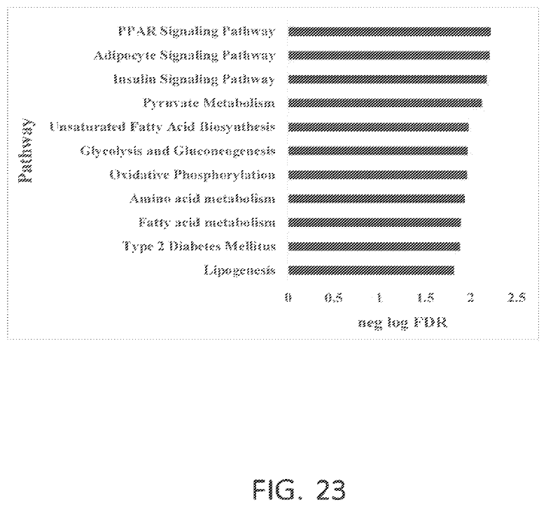

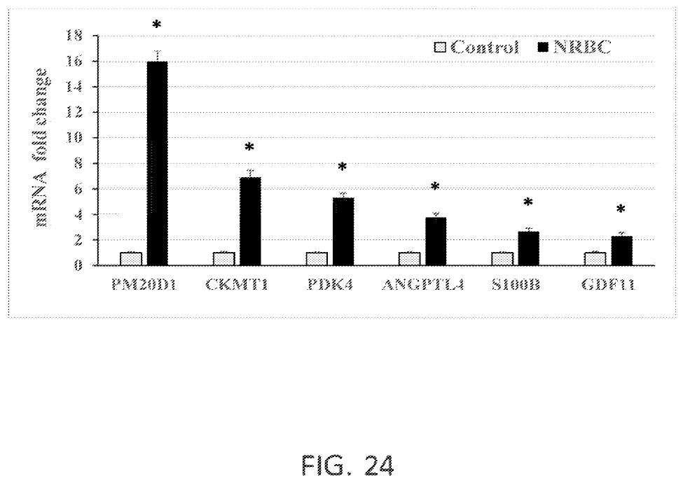

To expand our understanding of human adipocyte reprogramming by NRBC, we conducted whole transcriptome sequencing. Adipocyte cultures from two female donors with overweight and obesity were treated with cell medium (vehicle control) or NRBC for seven days and RNA samples were processed for library construction. Differential gene expression results were computed for 17525 genes of which 3881 genes were significantly regulated at an adjusted p-value <0.05. Pathway enrichment analysis identified PPAR signaling, adipocyte signaling and insulin signaling pathways to be the most significantly increased after treatment (FIG. 23). In addition, the data analysis showed increases in genes for metabolism of pyruvate, fatty acids, glucose, and amino acids. These changes in mRNA levels were validated for selected genes by qRT-PCR (FIG. 24, p<0001).

### 1. PPARg and PPARα Target Genes

NRBC robustly upregulated a number of classical brown and beige genes that have previously been identified as targets of synthetic PPARα and PPARγ ligands (10) including CIDEA, CITED, perilipins 2, -4, -5, PDK4, GK, AQP7, fatty acid elongases ELOVL 3, -5, -6, ACSL5 and the fatty acid binding proteins FABP3, FABP4 and FABP7 (Table 2). In addition, FABP5, which delivers retinoic acid to nuclear receptors was significantly upregulated.

TABLE 2
NRBC-induced genes associated with brown/beige phenotype
previously shown as upregulated by synthetic PPARγ
and PPARα activators

| Gene | Description | Fold | Padj* |
|---|---|---|---|
| CIDEA | cell death inducing DFFA like effector a | 12.4 | 4.04E−06 |
| CITED1 | Cbp/p300 interacting transactivator | 4.3 | 1.37E−11 |
| | domain 1 | | |
| PLIN2 | perilipin 2 | 1.8 | 2.56E−13 |
| PLIN4 | perilipin 4 | 4.1 | 4.16E−49 |
| PLIN5 | perilipin 5 | 3.0 | 1.39E−11 |
| AQP7 | aquaporin 7 glycerol channel | 3.5 | 4.26E−20 |
| ELOVL3 | ELOVL fatty acid elongase 3 | 2.4 | 5.38E−07 |
| ELOVL5 | ELOVL fatty acid elongase 5 | 2.2 | 2.46E−26 |
| ELOVL6 | ELOVL fatty acid elongase 6 | 2.3 | 5.71E−13 |
| ACSL5 | acyl-CoA synthetase long chain | 1.8 | 0.017 |
| | family member 5 | | |
| FABP3 | fatty acid binding protein 3 | 2.5 | 2.65E−14 |
| FABP4 | fatty acid binding protein 4 | 5.6 | 2.42E−89 |
| FABP7 | fatty acid binding protein 7 | 6.0 | 2.32E−09 |
| FABP5 | fatty acid binding protein 5 | 1.9 | 1.1E−05 |

*Adjusted p-value

### 2. Upregulation of Secreted Peptides and Adipokines

Adipose tissue is an endocrine organ that secretes proteins, hormones, and bioactive lipids with beneficial paracrine effects on whole-body fat and glucose metabolism. NRBC treatment significantly upregulated the expression of a number of these genes including ANGPTL4, FNDC4 and GDF11 (Table 3). ANGPTL4 is produced by adipocytes and promotes lipolysis (Table 3)(61). Both the full-length protein and a truncated form of ANGPTL4 are secreted, and their overexpression in mice stimulates energy expenditure, lowers adiposity, and converts white fat to the beige phenotype (62). FNDC4 induces UCP1 and beige genes and promotes insulin sensitivity in adipocytes (63, 64). GDF11, a circulating cytokine in the transforming growth factor β superfamily, declines with age and has been under investigation as an anti-aging therapeutic (65). Restoration of circulating levels in aged mice promotes adiponectin secretion by fat tissues and reduces adiposity (66).

TABLE 3
Genes encoding secreted lipokines
and peptides upregulated by NRBC

| Gene | Description | Fold | Padj* |
|---|---|---|---|
| ANGPTL4 | angiopoietin like 4 | 3.1 | 1.4E−34 |
| FNDC4 | fibronectin type III domain | 1.7 | 3.6E−08 |
| | containing 4 | | |
| GDF11 | growth differentiation factor 11 | 2.2 | 2.6E−24 |
| CYP4F11 | cytochrome P450 family 4 | 14.0 | 3.0E−05 |
| | subfamily F member 11 | | |
| EPHX1 | epoxide hydrolase 1 | 1.7 | 3.2E−06 |
| EPHX2 | epoxide hydrolase 2 | 1.6 | 6.2E−04 |
| NMB | neuromedin B | 2.7 | 1.9E−30 |
| POMC | proopiomelanocortin | 1.7 | 6.0E−06 |
| PCSK1 | proprotein convertase | 1.7 | 4.4E−03 |
| | subtilisin/kexin type 1 | | |
| S100B | S100 calcium binding protein B | 2.5 | 5.0E−11 |

*Adjusted p-value

Hydroxyeicosatetraenoic acids (HETEs) and dihydroxyoctadecanoic acids (diHOMEs) are secreted bioactive lipids that are produced from cytochrome P450 metabolites in brown adipocytes after cold exposure and exercise (67, 68). CYP4F11, a cytochrome that produces 20-HETE from arachidonic acid (69), was abundantly increased by NRBC. Circulating levels of 20-HETE correlate with elevated energy expenditure after cold exposure in people with detectable levels of BAT (70), and it is a PPARα activator (71). In addition, NRBC induced epoxide hydrolases EPHX1 and EPHX2, enzymes that utilize HETEs to produce 12,13-diHOME, a lipokine associated with improved insulin sensitivity and reduced triglycerides after exercise (72, 73).

Several circulating CNS-acting proteins were increased by NRBC. Neuromedin B is a peptide that acts on hypothalamic neurons to promote satiety, and a missense mutation is linked to hyperphagia and obesity in genetic studies (74, 75). Pro-opiomelanocortin (POMC), a peptide prohormone that is cleaved by PCSK1 into the hormone α-MSH, is involved in the suppression of food intake (76). POMC and PCSK1 were both elevated by NRBC in adipocytes. The important role of POMC is shown in studies linking severe obesity in humans to rare genetic mutations in POMC or PCSK1 (77). S100b, which has been characterized in brown adipocytes of mice, stimulates neurite outgrowth of sympathetic terminals in adipose tissue following cold exposure (78). The upregulation of genes for CNS-acting circulating factors indicates that appetite reduction can play a role in the physiological response to NRBC.

### 3. Increases in Thermogenesis Genes for Non-UCP1 Uncoupling and Substrate Cycling Pathways

Several mechanisms other than uncoupling of mitochondria by UCP1 can yield thermogenic energy expenditure. These mechanisms were originally identified in studies of brown adipose tissue (BAT) from cold exposed Ucp1-knockout mice that are able to maintain their body temperature (79). We found that NRBC induced significant increases in a number of new thermogenesis genes in human adipocytes (Table 4). RXRγ, a retinoic acid receptor isoform of unknown function, was highly upregulated. This isoform is localized in UCP1 positive cells in human adipose tissues and is expressed at elevated basal levels in adipose-derived stem cells that subsequently differentiate into UCP1-expressing brown adipocytes (80). These observations indicate that RXRγ can be a key transcription factor in the differentiation of human brown and beige adipocytes.

TABLE 4
Thermogenesis genes upregulated by NRBC

| Gene | Description | Fold | Padj* |
|---|---|---|---|
| RXRG | retinoid X receptor gamma | 10.0 | 8.2E−19 |
| PM20D1 | peptidase M20 domain containing 1 | 15.7 | 2.2E−07 |
| CKMT1A | creatine kinase, mitochondrial 1A | 5.1 | 3.7E−05 |
| CKMT1B | creatine kinase, mitochondrial 1B | 3.9 | 3.5E−06 |
| CKMT2 | creatine kinase, mitochondrial 2 | 3.8 | 5.1E−18 |
| PDK4 | pyruvate dehydrogenase kinase 4 | 4.2 | 1.0E−80 |
| GPD1 | glycerol-3-phosphate dehydrogenase 1 | 3.3 | 1.8E−25 |
| GK | glycerol kinase | 2.4 | 5.4E−03 |
| PCK1 | phosphoenolpyruvate carboxykinase 1 | 3.8 | 6.0E−08 |
| AIFM2 | apoptosis inducing factor mitochondria | 2.0 | 1.7E−29 |
| | associated 2 | | |
| UCP2 | uncoupling protein 2 | 3.4 | 2.5E−31 |
| NAMPT | nicotinamide phosphoribosyltransferase | 2.1 | 3.4E−23 |
| NAPRT | nicotinate phosphoribosyltransferase | 2.4 | 3.1E−22 |
| LIPE | hormone sensitive lipase | 3.2 | 2.5E−31 |
| PRKAR2B | Protein kinase cAMP-dep type II | 2.5 | 3.5E−20 |
| | regulatory subunit B | | |

*Adjusted p-value

PM20D1 levels were upregulated approximately 15-fold in adipocytes treated with NRBC, and human genetic studies have shown that the PM20D1 gene promoter has a PPRE (Benson et al., 2019). PM20D1 is a secreted enzyme that regulates synthesis and degradation of N-acyl amino acids (NAAs), molecules that directly uncouple mitochondria and increase energy expenditure (81). High PM20D1 levels in the white adipose tissue of mice correlates with increased respiration, reversal of high fat diet-induced obesity, and reductions in blood glucose (81, 82, 83). Creatine phosphate cycling is an enzymatic pathway that contributes to thermogenesis in Ucp1-knockout mice (84). The synthesis and breakdown of creatine phosphate by the mitochondrial creatine kinases CKMT1A, CKMT1B and CKMT2 releases heat and consumes ATP. We found strong upregulation of the genes for three CKMT isozymes after NRBC treatment of white adipocytes. In humans, CKMT proteins have previously only been detected in primary brown adipocytes (85).

Beige cells favor utilization of fatty acids rather than glucose to fuel thermogenesis and have high lipase activity (86). In addition to ATGL, hormone sensitive lipase (HSL) and protein kinase A regulatory subunit 2A (PRKAR2B) were upregulated by NRBC treatment (Table 4). PRKAR2B is the key PKA subunit regulating activation of lipolysis subsequent to ligand binding of Gs-coupled receptors (87). NRBC significantly upregulated genes for futile cycling of triglycerides, PDK4, GPD1, GK and PCK1. The activities of these enzymes result in shuttling of glycolytic intermediates into pyruvate for the synthesis of glycerol and glycerol-3-phosphate, the precursors for re-esterification of fatty acids during TG synthesis (10). NRBC also stimulated increases in AIFM2, an NADH oxidase (AIFM2) that supports glucose metabolism during thermogenesis in brown adipocytes (88), and UCP2. Elevated UCP2 levels are associated with cells that have high fatty acid oxidation rates, and evidence indicates that UCP2 facilitates fatty acid oxidation to fuel mitochondrial thermogenesis (89, 90, 91).

Nicotinamide phosphoribosyltransferase (NAMPT) and nicotinic acid phosphoribosyltransferase (NAPRT) encode enzymes that produce nicotine adenine dinucleotide (NAD) and are upregulated over two-fold. NAD is a key cofactor for cellular metabolism enzymes. Impaired NAD synthesis in adipocytes causes systemic insulin resistance and suppresses lipolysis and thermogenesis in mice (92, 93). In human adipocytes, increasing intracellular NAD induces UCP1 and mitochondrial biogenesis, which are markers of beige cells (94). In summary, these changes in thermogenesis genes indicate that NRBC-treated adipocytes have a higher capacity for uncoupled respiration and thermogenic futile cycling pathways.

4. NRBC Increases Expression of Receptors that Regulate Lipolysis and Thermogenesis

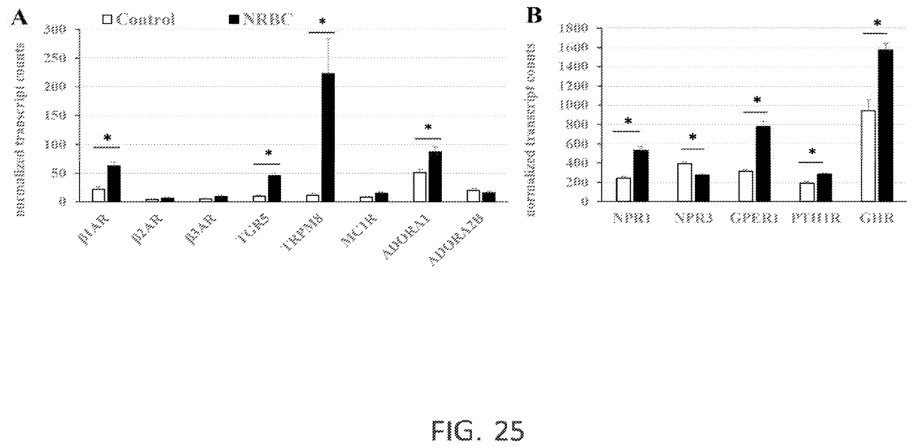

There is little comprehensive data available on relative receptor levels or hormone-stimulated lipolysis in human adipocytes. Based on our RNA sequencing data, receptor abundance was estimated from transcript reads per total number of kilobases sequenced (FIGS. 25a and 25b). NRBC treatment significantly increased expression of eight receptors potentially able to drive lipolysis or thermogenesis through various mechanisms including the B1AR, bile acid receptor TGR5, cold receptor TRPM8, adenosine receptor ADORA1, NPR1, G-protein coupled estrogen receptor-1 (GPER1), growth hormone receptor (GHR) and PTHR1 (Padj<0.002). In addition, there were increases in β2AR, β3AR, and melanocortin-1 receptor (MC1R) that did not achieve statistical significance due to variability in the response. The β2AR and β3 AR had the lowest expression levels compared to other receptors in white adipocytes. The β1AR was approximately four times more abundant than the β2AR and β3AR and increased another three-fold after NRBC exposure (FIG. 25a). TGR5, TRPM8 and ADORA1 and ADORA2 were also expressed at low levels in untreated white adipocytes.

The ratio of stimulatory NPR1 to the clearance receptor NPR3 determines the magnitude of the response to natriuretic peptides. NRBC treatment increased the NPR1/NPR3 ratio four-fold in NRBC-treated adipocytes, indicating that adipocytes can be more responsive to natriuretic peptides (FIG. 25b). PTHR levels were upregulated over two-fold by NRBC (FIG. 24b). GHR was the most abundant of the receptors upregulated by NRBC, and studies indicate that GH triggers lipolysis by unique mechanisms that occur downstream of receptor signaling in human adipocytes(41, 95). GPER1 is abundantly expressed in adipocytes and estradiol is the endogenous ligand. Little is known about the role of GPER1 in white adipocytes(96). A selective synthetic agonist for GPER stimulates weight loss and energy expenditure in mice(97).

NRBC-treated adipocytes have higher agonist-stimulated lipolysis than white adipocytes

Thermogenesis is fueled by lipolysis, so we determined whether agonist-stimulated glycerol release, a measure of lipolysis, reflected the increases in receptors and lipolysis machinery in NRBC-treated adipocytes. First, adipocyte cultures from four donors who had obesity were treated with cell medium (untreated control cells) or NRBC for seven days. On the day of the acute lipolysis assay, cells were exposed to the individual receptor agonists for four hours in buffer and glycerol released into the cell supernatant was measured. Non-hydrolyzable 8-cpt-cAMP, a potent activator of PKA signaling, was also evaluated in each experiment as a positive control since it bypasses individual receptors and measures the maximum capacity for lipolysis (36). The βARs, PTHR, MC1R, GPER and ADORA2B are all stimulatory G-protein (Gs) coupled receptors and signal through the CAMP-PKA signal transduction mechanism. NPR1 is not a Gs-coupled receptor and signals through an alternate mechanism of guanylate cyclase-cGMP-protein kinase G (PKG).

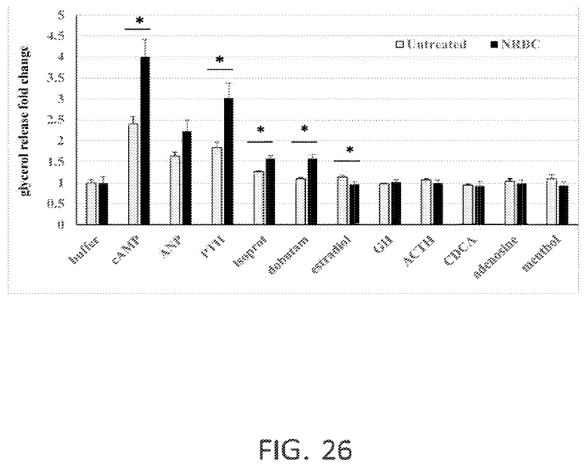

Of the hormones tested, PTH-stimulated lipolysis was the highest in control white adipocytes and increased by the greatest magnitude in cultures exposed to NRBC (FIG. 26). PTH treatment of human adipocytes was previously shown to stimulate UCP1 expression and oxygen consumption (98). We tested atrial natriuretic peptide (ANP), one of the key endogenous ligands for NPR1 released from atrial myocytes in response to increases in blood pressure and environmental stimuli such as exercise and cold exposure (99, 100). ANP-stimulated lipolysis increased in NRBC-treated cells, but the overall effect was not statistically significant in adipocytes from four donors.

The collective activity of the βARs was measured using isoproterenol, a non-selective agonist of the three βARs, to evaluate the overall potential for increased sensitivity to sympathetic stimulation. Since the β1AR was 5-fold more abundant than the other βARs, the β1AR-selective ligand dobutamine was also tested. Isoproterenol- and dobutamine-stimulated lipolysis were significantly boosted in NRBC-treated cells, and dobutamine activity was comparable with isoproterenol activity (FIG. 26). In NRBC-treated adipocytes, lipolysis stimulated by 8-cpt-cAMP was significantly higher than levels in control cells, showing that maximum capacity for lipolysis was elevated in parallel with the upregulation of the PKA regulatory subunit and lipases.

Agonists for the receptors GHR, MC1R, ADORA1/ADORA2B, TGR5 and TRPM8 did not stimulate detectable increases in lipolysis. In murine white and brown adipocytes adrenocorticotropic hormone (ACTH) and adenosine, endogenous ligands for melanocortin receptors and ADORA2B respectively, stimulate lipolysis and OCR (42, 44, 101). Bile acids activate TGR5 in human brown adipocytes and increase OCR (43). Although GPER activation by estrogen stimulates cAMP production in cancer cells (36), our data showed a small reduction in lipolysis.

### RXRγ is Bound to PPARγ Complexes Before and After Treatment

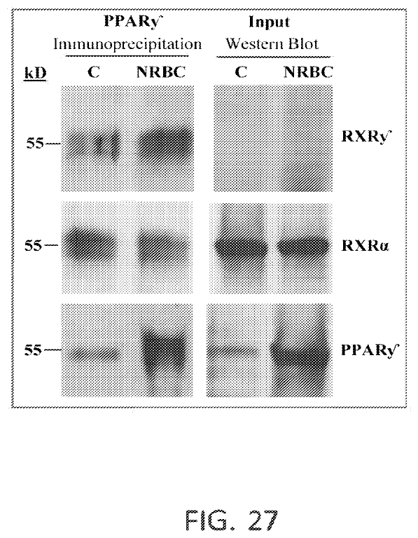

NRBC induced a strong increase in RXRγ mRNA after treatment and RXRα was unchanged. RXRγ is a unique isoform associated with differentiation of brown adipocytes in human adipose tissue (80). RXRs can form homodimers or heterodimers with multiple nuclear receptors other than PPARγ, so we evaluated whether RXRγ was bound to PPARγ after NRBC treatment. PPARγ was immunoprecipitated from protein lysates of untreated and NRBC-treated adipocytes. The protein complexes pulled down with PPARγ were analyzed on Western Blots (FIG. 27). RXRγ protein expression was too low for detection on regular Western Blots of whole cell protein, consistent with our transcriptome sequencing data indicating that it has a low copy number. However, we observed that RXRγ was visible in the immunoprecipitated PPARγ protein complex in control cells (white adipocytes) and in NRBC treated beige adipocytes (FIG. 27). RXRα is more abundantly expressed and was readily detected in Western Blots and in PPARγ immunoprecipitates. These results indicate that both isoforms are bound constitutively and that binding of RXRγ is independent of the addition of exogenous ligands. RXR isoforms stabilize PPARγ complexes at PPRE promoter elements and direct expression of specific target genes subsequent to ligand binding (46). Therefore, elevated levels of RXRγ can direct expression towards beige genes after the addition of BC.

### Discussion

Thermogenesis is a fundamental component of energy balance. A number of studies have demonstrated that human adipocytes have the functional plasticity to be transformed into thermogenic cells by non-adrenergic stimuli, making adipose tissue a relevant peripheral target tissue for obesity drugs (10, 37, 45) (98). In this report we investigated the potential of carotenoids to intensify the thermogenic response of human adipocytes to NR, a natural activator of PPARα and PPARγ. Pro-vitamin A carotenoids are converted into ligands for RXR nuclear receptors that form heterodimers with PPARs and coordinate expression of metabolism genes. We evaluated adipocytes treated for seven days with NR and carotenoids at concentrations that are reached in serum after oral administration to humans. The two pro-vitamin A carotenoids, BC and lutein, synergistically enhanced levels of UCP1 compared to NR alone. Lycopene, the third carotenoid that we investigated is not converted into an RXR ligand and did not alter the NR response. We used BC for additional experiments and found that NRBC also enhanced expression of ATGL, GLUT4, and adiponectin, which are drivers of lipolysis and insulin sensitivity. The effect of BC was selective. Expression of the PPARα target gene CPT1B, and PM20D1, a PPARγ regulated gene, were not elevated compared to treatment with NR alone.

NRBC upregulated protein levels of PGC-1α, PPARγ, and PPARα without comparable increases in mRNA levels, indicating that the mechanism does not involve enhanced translation. Pgc-1α is a cold-induced coactivator for PPARγ and PPARα with a short protein half-life, and its transcriptional activity is upregulated in mice by mechanisms that slow degradation (58). When its protein levels increase, Pgc-1α protein associates with the PPARγ/RXR complex and selectively promotes expression of UCP1 and mitochondrial proteins (102). Elevated PGC-1α protein drives the PPARα/RXR complex towards GK expression and TG cycling activity in human adipocytes (103). The half-life of PPARγ and PPARα proteins is regulated by control of degradation rate after binding of ligands and cofactors (56, 57, 59). Since treatment with NR alone did not upregulate protein levels, our data indicate that NR and BC are both required to stabilize PPAR protein levels.

Brown and beige adipocytes release specialized bioactive lipokines and proteins into circulation that activate whole body insulin sensitivity and fatty acid uptake. Transcriptome sequencing showed that NRBC treatment substantially upregulated Cyp4f11, EPDX1 and EPDX2, enzymes that produce HETES and di-HOMEs (69, 72). Secreted HETES and di-HOMEs can signal tissues to increase uptake of fatty acids, and these actions have beneficial effects on lowering blood lipids (73). HETEs are strong PPAR activators (71), and when combined with the increased levels of PPAR proteins observed after NRBC treatment can amplify PPAR target gene expression. NRBC also induced ANGPTL4, a secreted protein that stimulates adipocyte lipolysis, and the insulin sensitizers adiponectin, FNDC4, and GDF11.

ATGL is the rate-limiting lipase for release of fatty acids from TGs, and fatty acids are natural ligands of PPARα and PPARγ. In metabolically active beige cells, fatty acids are shuttled into mitochondria to fuel thermogenesis (86). In an energy-wasting futile cycle, fatty acids are also re-esterified onto the glycerol backbone of TGs. Whole transcriptome sequencing showed that NRBC stimulated a number of genes for glyceroneogenesis and TG synthesis, including PDK4, PCK1, GK, and GPD1. When thermogenesis is activated in adipocytes, PDK4 directs the flow of pyruvate generated by glycolysis into glycerol production (104). The PCK1 gene encodes PEPCK, the rate-limiting enzyme for glycerol synthesis from pyruvate. Studies using radio-labelled pyruvate in human adipocytes showed incorporation of the label into the glycerol backbone and into TGs after induction of PCK1 or PDK4 in human adipocytes (10, 105). In addition, GK and GPD1 encode enzymes that produce glycerol-3-phosphate, the substrate for fatty acid esterification (106, 107). The net result of TG cycling is elevated energy expenditure and a decrease of free fatty acids released into circulation (104).

In addition to UCP1 and TG cycling, we found that NRBC stimulated multiple other uncoupling and ATP-consuming enzymatic pathways. The most highly upregulated gene was PM20D1, encoding an enzyme that reversibly synthesizes NAAs that have potent mitochondrial uncoupling activity (108). In humans, the functional phenotype of PM20D1 is not well defined. In a clinical trial, serum concentrations of PM20D1 were positively correlated with adiposity and biomarkers of glucose metabolism such as glycated hemoglobin and fasting blood glucose (109). Both PM20D1 and NAAs are bound to serum proteins and regulation of circulating levels of NAAs in humans is complex (110). The relationship between adipose tissue PM20D1 and body weight in humans is unclear. Mutations in upstream PPARγ-binding regulatory elements of the PM20D1 gene cause a wide variation in basal expression levels in adipocytes. However, basal levels of PM20D1 in human adipose tissue do not correlate with circulating NAA levels (111). Presently, no clinical studies have been conducted to determine whether a substantial increase PM20D1 expression in thermogenic beige adipose tissue is sufficient to cause an increase in circulating NAA levels and contribute to weight loss.

The mitochondrial creatine kinases CKMT1A, CKMT1B and CKMT2 were highly induced by NRBC. Functional studies in mice showed that creatine kinases are upregulated in brown adipocytes after cold exposure or in the absence of Ucp1 and contribute to whole body energy expenditure (84). Interestingly, ablation of creatine metabolism in white adipose tissues inhibits thermogenesis and drives obesity in mice (112). In primary human brown adipocytes, proteomic analysis of thermogenesis pathways showed that ATP-coupled respiration is stimulated in parallel to uncoupled respiration and contributes half of the total oxygen consumed (85). Cycling of creatine phosphate supports ATP-coupled mitochondrial respiration by increasing the availability of ADP and phosphate to ATP-synthase. The strong induction of creatine kinases by NRBC indicates that ATP-coupled respiration and creatine phosphate cycling can contribute to thermogenesis in human beige adipocytes.

We showed that the enzymes involved in maintaining intracellular NAD levels, including AIFM2, NAMPT and NAPRT, increased after NRBC exposure. AIFM2 converts NADH to NAD to support the high levels required for glycolysis (88). NAMPT and NAPRT both synthesize NAD from intracellular precursors to support cellular needs during conditions of increased metabolic rate and oxidative stress (113). In addition, NAD is a cofactor for the sirtuin enzymes (SIRTs), which are protein deacetylases that regulate the activity of PPARγ, PGC-1α and other transcriptional activators of mitochondrial biogenesis and metabolism genes (114). Increasing NAD levels in human adipocytes can shift the phenotype from white to beige (94).

Our quantitative RNAseq analysis showed that the RXRα transcript is 300-fold more abundant than RXRγ in human white adipocytes, however, only the RXRγ isoform was robustly upregulated by NRBC treatment. We found that both RXRγ and RXRα proteins were bound in immunoprecipitated PPARγ complexes from untreated white adipocytes and in NRBC-treated beige cells. These results indicate that binding of both isoforms to the PPARγ complex is constitutive and ligand independent. In human perirenal adipose tissue, RXRγ expression is enriched in brown adipocyte progenitors and is induced in parallel with UCP1 during conversion of white to beige adipocytes (80). Possible mechanisms for targeting of beige genes can be recruitment of specific coactivators by RXRγ or conformational changes stimulated by BC binding that facilitate and stabilize interaction of this isoform with specific promoter elements (115) (46). In addition, the increase in RXRγ, PGC1α, PPARα and PPARγ proteins after NRBC treatment indicates the existence of a positive feedback loop that upregulates the genes identified in this study.

We used transcriptome sequencing data to estimate relative expression levels of receptors known to activate lipolysis or thermogenesis in adipocytes. In cells treated with NRBC, eight receptors were upregulated and several others trended higher. We evaluated stimulation of lipolysis with agonists for the receptors altered by NRBC and found that only a small subset were lipolytic. ACTH and adenosine stimulate lipolysis in murine white adipocytes and we saw no effect (44, 101). TGR5 and TRPM8 agonists induce thermogenesis genes in human adipocytes but did not stimulate lipolysis in our assay. We and others have shown that mild cold exposure of subcutaneous human adipocytes activates UCP1 expression by TRPM8, so the large increase in receptor levels after NRBC treatment has potential to act locally to sensitize adipocytes to cold and stimulate thermogenesis (45, 116). GHR and GPER1 were induced to the highest levels of the receptors after NRBC exposure, but neither growth hormone nor estrogen stimulated lipolysis.

NRBC upregulated PTHR levels. Moreover, PTH stimulated the greatest magnitude of lipolysis of the hormones tested in NRBC pretreated cells. PTH is released from parathyroid glands for regulation of systemic calcium homeostasis and all tissues express the PTHR(117). Evidence is growing that PTH plays an important role in adipose tissue metabolism. PTH stimulates lipolysis in mouse adipocytes and thermogenic gene expression in human adipocytes(98, 118). Cold-induced increases in circulating PTH shift whole-body metabolism toward lipid utilization to fuel energy expenditure in swimmers(119).

NRBC increased the stimulatory ratio of NPR1/NPR3 receptors. ANP is released after cold exposure and exercise and acts additively with adrenergic agonists to stimulate lipolysis and brown adipocyte characteristics in human white adipocytes(37). Although the increase was not statistically significant, our data showed a trend toward increased ANP-activated lipolysis in NRBC-treated cells.

We observed that the β1AR expression level in human adipocytes was substantially higher than the other βARs, and the β1AR was induced an additional three-fold by NRBC. We tested glycerol release with the pan-BAR agonist isoproterenol and the β1AR-selective agonist dobutamine and found that both stimulated a similar increase in NRBC-exposed cells compared to untreated. These results are in line with other studies showing that the β1AR is the dominant subtype in human white and brown adipocytes(39, 120).

NRBC significantly increased expression of PRKAR2B, the key PKA regulatory subunit linked to insulin sensitivity and resistance to weight gain in humans(87). We used 8-cpt-CAMP to activate PKA downstream of receptors and observed a considerable increase in the maximum capacity for lipolysis in adipocytes pretreated with NRBC. Impaired cAMP-stimulated TG lipolysis in subcutaneous adipose tissue is a characteristic of obesity and insulin resistance(121). A comprehensive analysis of two female cohorts with a ten-year follow-up was conducted to determine adipose tissue characteristics that predict weight gain. Low levels of stimulated lipolysis and PRKAR2B expression predicted weight gain and impaired glucose metabolism(122). Interestingly, there was no correlation with basal lipolysis or fat oxidation.

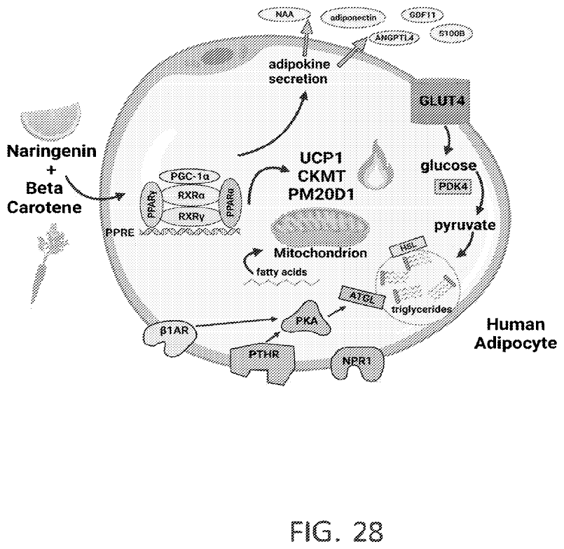

The human response to cold exposure and exercise involves transient release of ANP and PTH into circulation and sympathetic release of catecholamines in adipose tissues(99, 119)(37, 39, 98). Our data indicate that adipose tissue in an individual consuming NRBC can be more responsive to circulating hormones released after these stimuli. We demonstrate in this report that NRBC reprograms adipocytes by upregulating multiple thermogenic pathways, beneficial secreted factors, receptors and lipolysis, summarized in FIG. 28. NR and BC have a good safety profiles and have the potential to be administered long-term without adverse effects (27, 29). A randomized, double blinded placebo-controlled clinical trial will be needed to determine whether the effects of NRBC on adipocytes will translate into weight loss and improvements in insulin sensitivity.

### Materials and Methods

### Chemicals and Antibodies

Naringenin extract (NR) from whole Citrus sinensis oranges (purity≥30%) was purchased from GE Nutrients, Inc. (Gencor, Irvine, CA). BC, lycopene, and lutein were from Cayman Chemical Co. Protease and phosphatase inhibitors were purchased from Cell Signaling Technology (Danvers, MA), TGX protein gels from BIO-RAD (Hercules, CA). Type 1 collagenase, glycerol standard solution, adenosine, estradiol, human pituitary growth hormone, dobutamine hydrochloride, human atrial natriuretic peptide, ACTH, menthol, 8-CPT-CAMP were purchased from Sigma-Aldrich. Isoproterenol, human parathyroid hormone (1-34), CDCA, were purchased from Cayman Chemicals. Glycerol reagent A was from ZenBio (Durham, NC). All other chemicals were purchased from Sigma (St. Louis, Mo) unless otherwise indicated.

Primary antibodies used were UCP1 (#MAB6158, R&D Systems), GLUT4 (Ab654 Abcam), PGC-1α (ST1202, Sigma) and β-Actin (A5316, Sigma), or monoclonals from Santa Cruz against ATGL (sc-365278), adiponectin (sc-136131), PPARα (398394), PPARγ (sc-7273), NAMPT (sc-393444), RXRγ (sc-514134), RXRα (sc-515929). HRP-linked anti-rabbit (12-348, Sigma), anti-mouse (AP130P, Sigma) and anti-IgG kappa light chain (sc-516102, Santa Cruz) were used to detect specific antibody-antigen complexes. Western Lightning Plus-ECL was from PerkinElmer (Waltham, MA).

### Human Adipocyte Cell Culture

Human adipose-derived stem cells from overweight and obese female donors were purchased from LaCell, LLC (New Orleans, Louisiana) or isolated from lipoaspirate waste donated post-surgery from women with obesity using methods as previously described (31). Cells were seeded, maintained until two days after becoming confluent, and differentiated into adipocytes in the presence of rosiglitazone and isobutylmethylxanthine for five days as previously described (25). Treatments of adipocytes with 8 μM NR and 2 μM carotenoid, dissolved in DMSO at 1000×, started 5 days after the differentiation period and lasted for seven days in adipocyte maintenance medium with heat inactivated serum, before RNA and protein were isolated from adipocyte cultures.

### RNA Extraction and Transcriptome Sequencing

Total RNA was extracted from cells using Tri-reagent and purified with RNeasy (Qiagen) into nuclease-free water with RNAsecure Reagent (Thermo Fisher Scientific). RNA integrity was assessed using an Agilent Bioanalyzer 2100. RNA samples were diluted to 50 ng/μL and libraries were constructed using Lexogen Quant-Seq 3′ mRNA-Seq Library Prep Kit (SKU015.96) with oligo (dT) priming. Double-stranded cDNA was purified with magnetic beads, libraries were amplified using PCR, and transcripts were indexed, pooled, and forward-sequenced at 50 bp using NextSeq500 (Illumina). BlueBee software was used to analyze alignment and the DESeq2 V1.32.0 package in R V4.1.0, Rstudio V1.4.1717 and biomaRt V2.48.2 were used for differential expression analysis after estimation of possible outlier counts per gene via the Cook's distance, and their replacement by the trimmed mean over all counts for that gene. Differential gene expression results were computed for 17525 genes of which 3881 genes were differentially regulated at an adjusted p-value<0.05. Pathway enrichment analysis was carried out via the Gene Set Enrichment Analysis (GSEA) tool (32) by estimating enrichment on pathways present in the Kyoto Encyclopedia of Genes and Genomes (KEGG) database (33) available from the Molecular Signatures Database repository (MSigDb, http://software.broadinstitute.org/gsea/msigdb) (34), and additional custom pathways. Gene-sets with FDR≤5% were considered as significantly enriched (35).

Quantitative Real Time Polymerase Chain Reaction (qRT-PCR)

Quantitative reverse transcriptase and real-time PCR were conducted in one reaction with the gene-specific reverse PCR primer also priming the cDNA synthesis as previously described (25). Primer-probe sequences for UCP1, GLUT4, ATGL, adiponectin, PGC-1α and ribosomal RPL13A, used to adjust target gene values for total RNA in each sample, have been previously reported (25). Additional primer and probe oligonucleotide sets used in this study are shown in 5′ to 3′ orientation: PPARα Forward GTCGATTTCACAAGTGCCTTTC reverse CAGGTAAGAATTTCTGCTTTCAGTT probe AACGAATCGCGTTGTGTGACATCC; PDK4 forward CTGAGAATTATTGACCGCCTCT reverse GAAATTGGCAAGCCGTAACC probe TACATACTCCACTGCACCAACGCC; PPARγ forward CCCAAGTTTGAGTTTGCTGTG reverse GCGGTCTCCACTGAGAATAATG probe TGGAATTAGATGACAGCGACTTGGCA; NAMPT forward TGTTCCTTCAAGTGTAGCTATGT reverse TGCTGGCGTCCTATGTAAAG probe AACGTCTTCAAGGACCCAGTTGCT; CKMT1 forward CTTGACCTGTCCATCTAACCTG reverse ACTCCTCCAGTACCACGTT probe AGATAGCCGCTTCCCAAAGATCCTG. Predesigned human primer and probe sets from Thermofisher Scientific are PM20D1 Hs00399438_m1; ANGPTL4 Hs00211522_m1; GDF11 Hs00195156_m1; S100B Hs00902901_m1.

### Protein Extraction, PPARγ Immunoprecipitation and Western Blotting

Whole cell protein was isolated from differentiated cell cultures after treatment for seven days, lysed with RIPA buffer containing protease and phosphatase inhibitors, and pushed through a 20-gauge needle four times to disrupt organelles. Fifty micrograms of total cell protein were loaded per lane and resolved in 7.5% SDS-PAGE gels, transferred to nitrocellulose membranes, and probed overnight at 4° C. with specific primary antibodies. Anti-rabbit or -mouse secondary antibodies conjugated to horseradish peroxidase were used for detection of target proteins. Image J was used to quantify protein bands on Western blots. To adjust for variations in total protein loaded in each lane, β-actin was used.

PPARγ was immunoprecipitated from whole cell lysates with SC-7273 antibody (Santa Cruz) using the Pierce MS-compatible magnetic IP kit with protein A/G beads according to kit directions (Pierce 90409, Thermofisher). Briefly, primary anti-PPARγ antibody was incubated with 600 micrograms of adipocyte protein lysate for 4 hours at 4° C. on a tube inverter. The antibody-lysate mixture was then incubated with magnetic protein A/G beads for one hour, and complexes of PPARγ protein attached to beads were isolated, washed and eluted using a magnetic tube holder. Samples were evaporated to dryness under vacuum, resuspended in RIPA buffer and subjected to Western Blot analysis. An HRP-conjugated anti-IgG kappa light chain secondary antibody was used to eliminate primary antibody heavy chain bands from interfering with detection of immunoprecipitated target proteins. Proteins were visualized by chemiluminescence using Western Lightening (Amersham).

### Lipolysis Assay:

Cells were differentiated in 96-well plates and treated with cell medium (untreated) or NRBC for seven days. On the day of the lipolysis assay, cells were exposed for 4.5 hours to buffer (KRB with 1% BSA) or receptor agonists dissolved in buffer. Agonists were used at concentrations shown to give maximum responses and were: 8-Cpt-cAMP 200 μM non-hydrolyzable PKA activator of maximum stimulated lipolysis (cAMP) (36), atrial natriuretic peptide 0.1 μM for NPR1 and NPR3(37), parathyroid hormone (amino acids 1-34) 1 μM for PTHR(38), isoproterenol 1 μM all βARs and dobutamine 1 μM for β1AR (39), estradiol 1 μM for GPER (40), growth hormone 250 ng/ml for GHR (41), adrenocorticotropin hormone 1 μM for MC1R (42), bile acid chenodeoxycholic acid 30 μM for TGR5(43), adenosine 1 μM for ADORA1 and ADORA2B (44), and menthol 100 μM for TRPM8(45). Supernatants were removed for measurement of glycerol released using Glycerol Reagent A, and concentrations were determined using a standard curve. Data are from four or five experiments each with cells from different donors with BMI from 27 to 36.

### Statistical Analysis:

Statistical analyses were performed using SAS 9.4 (SAS Institute, Cary, North Carolina). The synergistic effect of the combination of NR and BC was investigated by implementing linear mixed effect models including plate as the random effect. The goal was to test if the combination (NRBC) induced a greater response compared to the sum of the individual components' effects. Based on the mixed effect model, an F test was constructed to evaluate the additive effect of NR over control and BC over control was no different from that of NRBC over control. We also used linear mixed effect models to test the effects of: 1) NR, BC, and NRBC compared to control for the target variables of mRNA and protein; 2) Agonist stimulated lipolysis in NRBC pre-treated cells v. untreated cells. Experiments were repeated at least three times in primary adipocytes from different donors. Significance was set at p<0.05. Data are reported as least squares means±standard error unless otherwise specified.

### REFERENCES CITED IN THIS EXAMPLE

1. Kajimura S, Spiegelman B M, Seale P. Brown and Beige Fat: Physiological Roles beyond Heat Generation. Cell Metab. 2015; 22(4): 546-59.
2. Guerra C, Koza R A, Yamashita H, Walsh K, Kozak L P. Emergence of brown adipocytes in white fat in mice is under genetic control. Effects on body weight and adiposity. J Clin Invest. 1998; 102(2): 412-20.
3. Kopecky J, Clarke G, Enerback S, Spiegelman B, Kozak L P. Expression of the mitochondrial uncoupling protein gene from the aP2 gene promoter prevents genetic obesity. J Clin Invest. 1995; 96(6): 2914-23.
4. Vallerand A L, Perusse F, Bukowiecki L J. Stimulatory effects of cold exposure and cold acclimation on glucose uptake in rat peripheral tissues. Am J Physiol. 1990; 259(5 Pt 2): R1043-9.
5. Barbatelli G, Murano I, Madsen L, Hao Q, Jimenez M, Kristiansen K, et al. The emergence of cold-induced brown adipocytes in mouse white fat depots is determined predominantly by white to brown adipocyte transdifferentiation. Am J Physiol Endocrinol Metab. 2010; 298(6): E1244-53.
6. Himms-Hagen J, Cui J, Danforth E, Jr., Taatjes D J, Lang S S, Waters B L, et al. Effect of CL-316,243, a thermogenic beta 3-agonist, on energy balance and brown and white adipose tissues in rats. Am J Physiol. 1994; 266(4 Pt 2): R1371-82.
7. Cypess A M, Chen Y C, Sze C, Wang K, English J, Chan O, et al. Cold but not sympathomimetics activates human brown adipose tissue in vivo. Proc Natl Acad Sci USA. 2012; 109(25): 10001-5.
8. Finlin B S, Memetimin H, Zhu B, Confides A L, Vekaria H J, El Khouli R H, et al. The beta3-adrenergic receptor agonist mirabegron improves glucose homeostasis in obese humans. J Clin Invest. 2020; 130(5): 2319-31.
9 Vosselman M J, van der Lans A A, Brans B, Wierts R, van Baak M A, Schrauwen P, et al. Systemic beta-adrenergic stimulation of thermogenesis is not accompanied by brown adipose tissue activity in humans. Diabetes. 2012; 61(12): 3106-13.
10. Barquissau V, Beuzelin D, Pisani D F, Beranger G E, Mairal A, Montagner A, et al. White-to-brite conversion in human adipocytes promotes metabolic reprogramming towards fatty acid anabolic and catabolic pathways. Mol Metab. 2016; 5(5): 352-65.
11. Loft A, Forss I, Siersbaek M S, Schmidt S F, Larsen A S, Madsen J G, et al. Browning of human adipocytes requires KLF11 and reprogramming of PPARgamma superenhancers. Genes Dev. 2015; 29(1): 7-22.
12. Kaupang A, Hansen T V. The PPAR Omega Pocket: Renewed Opportunities for Drug Development. PPAR Res. 2020; 2020:9657380.
13. Tan Y, Muise E S, Dai H, Raubertas R, Wong K K, Thompson G M, et al. Novel transcriptome profiling analyses demonstrate that selective peroxisome proliferator-activated receptor gamma (PPARgamma) modulators display attenuated and selective gene regulatory activity in comparison with PPARgamma full agonists. Mol Pharmacol. 2012; 82(1): 68-79.
14. Benvenuti S, Cellai I, Luciani P, Deledda C, Baglioni S, Giuliani C, et al. Rosiglitazone stimulates adipogenesis and decreases osteoblastogenesis in human mesenchymal stem cells. J Endocrinol Invest. 2007; 30(9): RC26-30.
15. Soccio R E, Chen E R, Lazar M A. Thiazolidinediones and the promise of insulin sensitization in type 2 diabetes. Cell Metab. 2014; 20(4): 573-91.
16. Ribet C, Montastier E, Valle C, Bezaire V, Mazzucotelli A, Mairal A, et al. Peroxisome proliferator-activated receptor-alpha control of lipid and glucose metabolism in human white adipocytes. Endocrinology. 2010; 151(1): 123-33.
17. Allen L H. How common is vitamin B-12 deficiency? Am J Clin Nutr. 2009; 89(2): 693S-6S.
18. FDA.gov. Tricor fenofibrate [Available from: https://www.accessdata.fda.gov/drugsatfda_docs/label/2010/021656s019lbl.pdf.
19. Cheng H S, Tan W R, Low Z S, Marvalim C, Lee J Y H, Tan N S. Exploration and Development of PPAR Modulators in Health and Disease: An Update of Clinical Evidence. Int J Mol Sci. 2019; 20(20).
20 Boeckmans J, Natale A, Rombaut M, Buyl K, Rogiers V, De Kock J, et al. Anti-NASH Drug Development Hitches a Lift on PPAR Agonism. Cells. 2019; 9(1).
21. Steele H, Gomez-Duran A, Pyle A, Hopton S, Newman J, Stefanetti R J, et al. Metabolic effects of bezafibrate in mitochondrial disease. EMBO Mol Med. 2020; 12(3): e11589.
22 Choi J H, Banks A S, Estall J L, Kajimura S, Bostrom P, Laznik D, et al. Anti-diabetic drugs inhibit obesity-linked phosphorylation of PPARgamma by Cdk5. Nature. 2010; 466(7305): 451-6.
23 Zhang F, Lavan B E, Gregoire F M. Selective Modulators of PPAR-gamma Activity: Molecular Aspects Related to Obesity and Side-Effects. PPAR Res. 2007; 2007:32696.
24. Goldwasser J, Cohen P Y, Yang E, Balaguer P, Yarmush M L, Nahmias Y. Transcriptional regulation of human and rat hepatic lipid metabolism by the grapefruit flavonoid naringenin: role of PPARalpha, PPARgamma and LXRalpha. PLOS One. 2010; 5(8): e12399.
25. Rebello C J, Greenway F L, Lau F H, Lin Y, Stephens J M, Johnson W D, et al. Naringenin Promotes Thermogenic Gene Expression in Human White Adipose Tissue. Obesity (Silver Spring). 2019; 27(1): 103-11.
26 Murugesan N, Woodard K, Ramaraju R, Greenway F L, Coulter A A, Rebello C J. Naringenin Increases Insulin Sensitivity and Metabolic Rate: A Case Study. J Med Food. 2020; 23(3): 343-8.
27. Rebello C J, Beyl R A, Lertora J J L, Greenway F L, Ravussin E, Ribnicky D M, et al. Safety and pharmacokinetics of naringenin: A randomized, controlled, single-ascending-dose clinical trial. Diabetes Obes Metab. 2020; 22(1): 91-8.
28. Canas J A. Mixed carotenoid supplementation and dysmetabolic obesity: gaps in knowledge. Int J Food Sci Nutr. 2021; 72(5): 653-9.
29 Canas J A, Lochrie A, McGowan A G, Hossain J, Schettino C, Balagopal P B. Effects of Mixed Carotenoids on Adipokines and Abdominal Adiposity in Children: A Pilot Study. J Clin Endocrinol Metab. 2017; 102(6): 1983-90.
30. Coulter A A, Rebello C J, Greenway F L. Centrally Acting Agents for Obesity: Past, Present, and Future. Drugs. 2018; 78(11): 1113-32.
31. Li J, Curley J L, Floyd Z E, Wu X, Halvorsen Y D C, Gimble J M. Isolation of Human Adipose-Derived Stem Cells from Lipoaspirates. Methods Mol Biol. 2018; 1773:155-65.
32 Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43): 15545-50.
33. Ogata H, Goto S, Sato K, Fujibuchi W, Bono H, Kanehisa M. KEGG: Kyoto Encyclopedia of Genes and Genomes. Nucleic Acids Res. 1999; 27(1): 29-34.
34. Liberzon A, Birger C, Thorvaldsdottir H, Ghandi M, Mesirov J P, Tamayo P. The Molecular Signatures Database (MSigDB) hallmark gene set collection. Cell Syst. 2015; 1(6): 417-25.
35. Reiner A, Yekutieli D, Benjamini Y. Identifying differentially expressed genes using false discovery rate controlling procedures. Bioinformatics. 2003; 19(3): 368-75.
36. Gettys T W, Blackmore P F, Corbin J D. An assessment of phosphodiesterase activity in situ after treatment of hepatocytes with hormones. Am J Physiol. 1988; 254(4 Pt 1): E449-53.
37. Bordicchia M, Liu D, Amri E Z, Ailhaud G, Dessi-Fulgheri P, Zhang C, et al. Cardiac natriuretic peptides act via p38 MAPK to induce the brown fat thermogenic program in mouse and human adipocytes. J Clin Invest. 2012; 122(3): 1022-36.
38. Hoare S R, Gardella T J, Usdin T B. Evaluating the signal transduction mechanism of the parathyroid hormone 1 receptor. Effect of receptor-G-protein interaction on the ligand binding mechanism and receptor conformation. J Biol Chem. 2001; 276(11): 7741-53.
39. Riis-Vestergaard M J, Richelsen B, Bruun J M, Li W, Hansen J B, Pedersen S B. Beta-1 and Not Beta-3 Adrenergic Receptors May Be the Primary Regulator of Human Brown Adipocyte Metabolism. J Clin Endocrinol Metab. 2020; 105(4).
40. Filardo E J, Quinn J A, Frackelton A R, Jr., Bland K I. Estrogen action via the G protein-coupled receptor, GPR30: stimulation of adenylyl cyclase and cAMP-mediated attenuation of the epidermal growth factor receptor-to-MAPK signaling axis. Mol Endocrinol. 2002; 16(1): 70-84.
41. Sharma V M, Vestergaard E T, Jessen N, Kolind-Thomsen P, Nellemann B, Nielsen T S, et al. Growth hormone acts along the PPARgamma-FSP27 axis to stimulate lipolysis in human adipocytes. Am J Physiol Endocrinol Metab. 2019; 316(1): E34-E42.
42. Schnabl K, Westermeier J, Li Y, Klingenspor M. Opposing Actions of Adrenocorticotropic Hormone and Glucocorticoids on UCP1-Mediated Respiration in Brown Adipocytes. Front Physiol. 2018; 9:1931.
43. Broeders E P, Nascimento E B, Havekes B, Brans B, Roumans K H, Tailleux A, et al. The Bile Acid Chenodeoxycholic Acid Increases Human Brown Adipose Tissue Activity. Cell Metab. 2015; 22(3): 418-26.
44. Gnad T, Scheibler S, von Kugelgen I, Scheele C, Kilic A, Glode A, et al. Adenosine activates brown adipose tissue and recruits beige adipocytes via A2A receptors. Nature. 2014; 516(7531): 395-9.
45. Rossato M, Granzotto M, Macchi V, Porzionato A, Petrelli L, Calcagno A, et al. Human white adipocytes express the cold receptor TRPM8 which activation induces UCP1 expression, mitochondrial activation and heat production. Mol Cell Endocrinol. 2014; 383(1-2): 137-46.
46. A I, Tan N S, Gelman L, Kersten S, Seydoux J, Xu J, et al. In vivo activation of PPAR target genes by RXR homodimers. EMBO J. 2004; 23(10): 2083-91.
47. Rao A V, Rao L G. Carotenoids and human health. Pharmacol Res. 2007; 55(3): 207-16.
48. Pierce J P, Natarajan L, Sun S, Al-Delaimy W, Flatt S W, Kealey S, et al. Increases in plasma carotenoid concentrations in response to a major dietary change in the women's healthy eating and living study. Cancer Epidemiol Biomarkers Prev. 2006; 15(10): 1886-92.
49. Arballo J, Amengual J, Erdman J W, Jr. Lycopene: A Critical Review of Digestion, Absorption, Metabolism, and Excretion. Antioxidants (Basel). 2021; 10(3).
50. Unlu N Z, Bohn T, Francis D, Clinton S K, Schwartz S J. Carotenoid absorption in humans consuming tomato sauces obtained from tangerine or high-beta-carotene varieties of tomatoes. J Agric Food Chem. 2007; 55(4): 1597-603.
51. Bezaire V, Mairal A, Ribet C, Lefort C, Girousse A, Jocken J, et al. Contribution of adipose triglyceride lipase and hormone-sensitive lipase to lipolysis in hMADS adipocytes. J Biol Chem. 2009; 284(27): 18282-91.
52. Moraes-Vieira P M, Saghatelian A, Kahn B B. GLUT4 Expression in Adipocytes Regulates De Novo Lipogenesis and Levels of a Novel Class of Lipids With Antidiabetic and Anti-inflammatory Effects. Diabetes. 2016; 65(7): 1808-15.
53. Carvalho E, Kotani K, Peroni O D, Kahn B B. Adipose-specific overexpression of GLUT4 reverses insulin resistance and diabetes in mice lacking GLUT4 selectively in muscle. Am J Physiol Endocrinol Metab. 2005; 289(4): E551-61.
54. Achari A E, Jain S K. Adiponectin, a Therapeutic Target for Obesity, Diabetes, and Endothelial Dysfunction. Int J Mol Sci. 2017; 18(6).
55. Miller K N, Clark J P, Anderson R M. Mitochondrial regulator PGC-1α-Modulating the modulator. Curr Opin Endocr Metab Res. 2019; 5:37-44.
56. Kilroy G E, Zhang X, Floyd Z E. PPAR-gamma AF-2 domain functions as a component of a ubiquitin-dependent degradation signal. Obesity (Silver Spring). 2009; 17(4): 665-73.
57 Blanquart C, Barbier O, Fruchart J C, Staels B, Glineur C. Peroxisome proliferator-activated receptor alpha (PPARalpha) turnover by the ubiquitin-proteasome system controls the ligand-induced expression level of its target genes. J Biol Chem. 2002; 277(40): 37254-9.
58. Wei P, Pan D, Mao C, Wang Y X. RNF34 is a cold-regulated E3 ubiquitin ligase for PGC-1alpha and modulates brown fat cell metabolism. Mol Cell Biol. 2012; 32(2): 266-75.
59. Blanquart C, Mansouri R, Fruchart J C, Staels B, Glineur C. Different ways to regulate the PPARalpha stability. Biochem Biophys Res Commun. 2004; 319(2): 663-70.
60. Hauser S, Adelmant G, Sarraf P, Wright H M, Mueller E, Spiegelman B M. Degradation of the peroxisome proliferator-activated receptor gamma is linked to ligand-dependent activation. J Biol Chem. 2000; 275(24): 18527-33.
61. Gray N E, Lam L N, Yang K, Zhou A Y, Koliwad S, Wang J C. Angiopoietin-like 4 (Angptl4) protein is a physiological mediator of intracellular lipolysis in murine adipocytes. J Biol Chem. 2017; 292(39): 16135.
62. McQueen A E, Kanamaluru D, Yan K, Gray N E, Wu L, Li M L, et al. The C-terminal fibrinogen-like domain of angiopoietin-like 4 stimulates adipose tissue lipolysis and promotes energy expenditure. J Biol Chem. 2017; 292(39): 16122-34.
63. Fruhbeck G, Fernandez-Quintana B, Paniagua M, Hernandez-Pardos A W, Valenti V, Moncada R, et al. FNDC4, a novel adipokine that reduces lipogenesis and promotes fat browning in human visceral adipocytes. Metabolism. 2020; 108:154261.
64. Georgiadi A, Lopez-Salazar V, Merahbi R E, Karikari R A, Ma X, Mourao A, et al. Orphan GPR116 mediates the insulin sensitizing effects of the hepatokine FNDC4 in adipose tissue. Nat Commun. 2021; 12(1): 2999.
65. Fadini G P, Menegazzo L, Bonora B M, Mazzucato M, Persano S, Vigili de Kreutzenberg S, et al. Effects of Age, Diabetes, and Vascular Disease on Growth Differentiation Factor 11: First-in-Human Study. Diabetes Care. 2015; 38(8): e118-9.
66. Katsimpardi L, Kuperwasser N, Camus C, Moigneu C, Chiche A, Tolle V, et al. Systemic GDF11 stimulates the secretion of adiponectin and induces a calorie restriction-like phenotype in aged mice. Aging Cell. 2020; 19(1): e13038.
67. Lynes M D, Leiria L O, Lundh M, Bartelt A, Shamsi F, Huang T L, et al. The cold-induced lipokine 12, 13-diHOME promotes fatty acid transport into brown adipose tissue. Nat Med. 2017; 23(5): 631-7.
68. Gollasch B, Dogan I, Rothe M, Gollasch M, Luft F C. Maximal exercise and plasma cytochrome P450 and lipoxygenase mediators: a lipidomics study. Physiol Rep. 2019; 7(13): e14165.
69. Yi M, Cho S A, Min J, Kim D H, Shin J G, Lee S J. Functional characterization of a common CYP4F11 genetic variant and identification of functionally defective CYP4F11 variants in erythromycin metabolism and 20-HETE synthesis. Arch Biochem Biophys. 2017; 620:43-51.
70. Kulterer O C, Niederstaetter L, Herz C T, Haug A R, Bileck A, Pils D, et al. The Presence of Active Brown Adipose Tissue Determines Cold-Induced Energy Expenditure and Oxylipin Profiles in Humans. J Clin Endocrinol Metab. 2020; 105(7).
71. Ng V Y, Huang Y, Reddy L M, Falck J R, Lin E T, Kroetz D L. Cytochrome P450 eicosanoids are activators of peroxisome proliferator-activated receptor alpha. Drug Metab Dispos. 2007; 35(7): 1126-34.
72. Stanford K I, Lynes M D, Takahashi H, Baer L A, Arts P J, May F J, et al. 12, 13-diHOME: An Exercise-Induced Lipokine that Increases Skeletal Muscle Fatty Acid Uptake. Cell Metab. 2018; 27(6): 1357.
73. Vasan S K, Noordam R, Gowri M S, Neville M J, Karpe F, Christodoulides C. The proposed systemic thermogenic metabolites succinate and 12,13-diHOME are inversely associated with adiposity and related metabolic traits: evidence from a large human cross-sectional study. Diabetologia. 2019; 62(11): 2079-87.
74. Bouchard L, Drapeau V, Provencher V, Lemieux S, Chagnon Y, Rice T, et al. Neuromedin beta: a strong candidate gene linking eating behaviors and susceptibility to obesity. Am J Clin Nutr. 2004; 80(6): 1478-86.
75. Blanchet R, Lemieux S, Couture P, Bouchard L, Vohl M C, Perusse L. Effects of neuromedin-beta on caloric compensation, eating behaviours and habitual food intake. Appetite. 2011; 57(1): 21-7.
76. Schwartz M W, Woods S C, Porte D, Jr., Seeley R J, Baskin D G. Central nervous system control of food intake. Nature. 2000; 404(6778): 661-71.
77. Wabitsch M, Farooqi S, Fluck C E, Bratina N, Mallya U G, Stewart M, et al. Natural History of Obesity Due to POMC, PCSK1, and LEPR Deficiency and the Impact of Setmelanotide. J Endocr Soc. 2022; 6(6): bvac057.
78. Zeng X, Ye M, Resch J M, Jedrychowski M P, Hu B, Lowell B B, et al. Innervation of thermogenic adipose tissue via a calsyntenin 3beta-S100b axis. Nature. 2019; 569(7755): 229-35.
79. Chang S H, Song N J, Choi J H, Yun U J, Park K W. Mechanisms underlying UCP1 dependent and independent adipocyte thermogenesis. Obes Rev. 2019; 20(2): 241-51.
80. Svensson P A, Lindberg K, Hoffmann J M, Taube M, Pereira M J, Mohsen-Kanson T, et al. Characterization of brown adipose tissue in the human perirenal depot. Obesity (Silver Spring). 2014; 22(8): 1830-7.
81. Long J Z, Svensson K J, Bateman L A, Lin H, Kamenecka T, Lokurkar I A, et al. The Secreted Enzyme PM20D1 Regulates Lipidated Amino Acid Uncouplers of Mitochondria. Cell. 2016; 166(2): 424-35.
82. Long J Z, Roche A M, Berdan C A, Louie S M, Roberts A J, Svensson K J, et al. Ablation of PM20D1 reveals N-acyl amino acid control of metabolism and nociception. Proc Natl Acad Sci USA. 2018; 115(29):E6937-E45.
83. Li D, Liu Y, Gao W, Han J, Yuan R, Zhang M, et al. Inhibition of miR-324-5p increases PM20D1-mediated white and brown adipose loss and reduces body weight in juvenile mice. Eur J Pharmacol. 2019; 863:172708.
84. Kazak L, Chouchani E T, Jedrychowski M P, Erickson B K, Shinoda K, Cohen P, et al. A creatine-driven substrate cycle enhances energy expenditure and thermogenesis in beige fat. Cell. 2015; 163(3): 643-55.
85. Muller S, Balaz M, Stefanicka P, Varga L, Amri E Z, Ukropec J, et al. Proteomic Analysis of Human Brown Adipose Tissue Reveals Utilization of Coupled and Uncoupled Energy Expenditure Pathways. Sci Rep. 2016; 6:30030.
86. Braun K, Oeckl J, Westermeier J, Li Y, Klingenspor M. Non-adrenergic control of lipolysis and thermogenesis in adipose tissues. J Exp Biol. 2018; 221 (Pt Suppl 1).
87. Mantovani G, Bondioni S, Alberti L, Gilardini L, Invitti C, Corbetta S, et al. Protein kinase A regulatory subunits in human adipose tissue: decreased R2B expression and activity in adipocytes from obese subjects. Diabetes. 2009; 58(3): 620-6.
88. Nguyen H P, Yi D, Lin F, Viscarra J A, Tabuchi C, Ngo K, et al. Aifm2, a NADH Oxidase, Supports Robust Glycolysis and Is Required for Cold- and Diet-Induced Thermogenesis. Mol Cell. 2020; 77(3): 600-17 e4.
89. Caron A, Labbe S M, Carter S, Roy M C, Lecomte R, Ricquier D, et al. Loss of UCP2 impairs cold-induced non-shivering thermogenesis by promoting a shift toward glucose utilization in brown adipose tissue. Biochimie. 2017; 134:118-26.
90. Bouillaud F. UCP2, not a physiologically relevant uncoupler but a glucose sparing switch impacting ROS production and glucose sensing. Biochim Biophys Acta. 2009; 1787(5): 377-83.
91. Jahansouz C, Xu H, Hertzel A V, Kizy S, Steen K A, Foncea R, et al. Partitioning of adipose lipid metabolism by altered expression and function of PPAR isoforms after bariatric surgery. Int J Obes (Lond). 2018; 42(2): 139-46.
92. Yamaguchi S, Franczyk M P, Chondronikola M, Qi N, Gunawardana S C, Stromsdorfer K L, et al. Adipose tissue NAD (+) biosynthesis is required for regulating adaptive thermogenesis and whole-body energy homeostasis in mice. Proc Natl Acad Sci USA. 2019; 116(47): 23822-8.
93. Yamaguchi S, Yoshino J. Adipose tissue NAD (+) biology in obesity and insulin resistance: From mechanism to therapy. Bioessays. 2017; 39(5).
94 Nagy L, Rauch B, Szerafin T, Uray K, Toth A, Bai P. Nicotinamide-riboside shifts the differentiation of human primary white adipocytes to beige adipocytes impacting substrate preference and uncoupling respiration through SIRT1 activation and mitochondria-derived reactive species production. Front Cell Dev Biol. 2022; 10:979330.
95. Balaz M, Ukropcova B, Kurdiova T, Vlcek M, Surova M, Krumpolec P, et al. Improved adipose tissue metabolism after 5-year growth hormone replacement therapy in growth hormone deficient adults: The role of zinc-alpha2-glycoprotein. Adipocyte. 2015; 4(2): 113-22.
96. Prossnitz E R, Barton M. Estrogen biology: new insights into GPER function and clinical opportunities. Mol Cell Endocrinol. 2014; 389(1-2): 71-83.
97. Sharma G, Hu C, Staquicini D I, Brigman J L, Liu M, Mauvais-Jarvis F, et al. Preclinical efficacy of the GPER-selective agonist G-1 in mouse models of obesity and diabetes. Sci Transl Med. 2020; 12(528).
98. Hedesan O C, Fenzl A, Digruber A, Spirk K, Baumgartner-Parzer S, Bilban M, et al. Parathyroid hormone induces a browning program in human white adipocytes. Int J Obes (Lond). 2019; 43(6): 1319-24.
99. Carper D, Coue M, Nascimento EBM, Barquissau V, Lagarde D, Pestourie C, et al. Atrial Natriuretic Peptide Orchestrates a Coordinated Physiological Response to Fuel Non-shivering Thermogenesis. Cell Rep. 2020; 32(8): 108075.
100. Moro C, Crampes F, Sengenes C, De Glisezinski I, Galitzky J, Thalamas C, et al. Atrial natriuretic peptide contributes to physiological control of lipid mobilization in humans. FASEB J. 2004; 18(7): 908-10.
101. Cho K J, Shim J H, Cho M C, Choe Y K, Hong J T, Moon D C, et al. Signaling pathways implicated in alpha-melanocyte stimulating hormone-induced lipolysis in 3T3-L1 adipocytes. J Cell Biochem. 2005; 96(4): 869-78.
102. Puigserver P, Wu Z, Park C W, Graves R, Wright M, Spiegelman B M. A cold-inducible coactivator of nuclear receptors linked to adaptive thermogenesis. Cell. 1998; 92(6): 829-39.
103. Mazzucotelli A, Viguerie N, Tiraby C, Annicotte J S, Mairal A, Klimcakova E, et al. The transcriptional coactivator peroxisome proliferator activated receptor (PPAR) gamma coactivator-1 alpha and the nuclear receptor PPAR alpha control the expression of glycerol kinase and metabolism genes independently of PPAR gamma activation in human white adipocytes. Diabetes. 2007; 56(10): 2467-75.
104. Cadoudal T, Distel E, Durant S, Fouque F, Blouin J M, Collinet M, et al. Pyruvate dehydrogenase kinase 4: regulation by thiazolidinediones and implication in glyceroneogenesis in adipose tissue. Diabetes. 2008; 57(9): 2272-9.
105. Cadoudal T, Blouin J M, Collinet M, Fouque F, Tan G D, Loizon E, et al. Acute and selective regulation of glyceroneogenesis and cytosolic phosphoenolpyruvate carboxykinase in adipose tissue by thiazolidinediones in type 2 diabetes. Diabetologia. 2007; 50(3): 666-75.
106. Iwase M, Tokiwa S, Seno S, Mukai T, Yeh Y S, Takahashi H, et al. Glycerol kinase stimulates uncoupling protein 1 expression by regulating fatty acid metabolism in beige adipocytes. J Biol Chem. 2020; 295(20): 7033-45.
107. Ou X, Ji C, Han X, Zhao X, Li X, Mao Y, et al. Crystal structures of human glycerol 3-phosphate dehydrogenase 1 (GPD1). J Mol Biol. 2006; 357(3): 858-69.
108. Keipert S, Kutschke M, Ost M, Schwarzmayr T, van Schothorst E M, Lamp D, et al. Long-Term Cold Adaptation Does Not Require FGF21 or UCP1. Cell Metab. 2017; 26(2): 437-46 e5.
109. Yang R, Hu Y, Lee C H, Liu Y, Diaz-Canestro C, Fong C H Y, et al. PM20D1 is a circulating biomarker closely associated with obesity, insulin resistance and metabolic syndrome. Eur J Endocrinol. 2021; 186(2): 151-61.
110. Kim J T, Jedrychowski M P, Wei W, Fernandez D, Fischer C R, Banik S M, et al. A Plasma Protein Network Regulates PM20D1 and N-Acyl Amino Acid Bioactivity. Cell Chem Biol. 2020; 27(9): 1130-9 e4.
111. Benson K K, Hu W, Weller A H, Bennett A H, Chen E R, Khetarpal S A, et al. Natural human genetic variation determines basal and inducible expression of PM20D1, an obesity-associated gene. Proc Natl Acad Sci USA. 2019; 116(46): 23232-42.
112. Kazak L, Chouchani E T, Lu G Z, Jedrychowski M P, Bare C J, Mina A I, et al. Genetic Depletion of Adipocyte Creatine Metabolism Inhibits Diet-Induced Thermogenesis and Drives Obesity. Cell Metab. 2017; 26(4): 660-71 e3.
113. Audrito V, Messana V G, Deaglio S. NAMPT and NAPRT: Two Metabolic Enzymes With Key Roles in Inflammation. Front Oncol. 2020; 10:358.
114. Qiang L, Wang L, Kon N, Zhao W, Lee S, Zhang Y, et al. Brown remodeling of white adipose tissue by SirT1-dependent deacetylation of Ppargamma. Cell. 2012; 150(3): 620-32.
115. DiRenzo J, Soderstrom M, Kurokawa R, Ogliastro M H, Ricote M, Ingrey S, et al. Peroxisome proliferator-activated receptors and retinoic acid receptors differentially control the interactions of retinoid X receptor heterodimers with ligands, coactivators, and corepressors. Mol Cell Biol. 1997; 17(4): 2166-76.
116. Rebello C J, Coulter A A, Reaume A G, Cong W, Cusimano L A, Greenway F L. MLR-1023 Treatment in Mice and Humans Induces a Thermogenic Program, and Menthol Potentiates the Effect. Pharmaceuticals (Basel). 2021; 14(11).
117. Wysolmerski J J, Stewart A F. The physiology of parathyroid hormone-related protein: an emerging role as a developmental factor. Annu Rev Physiol. 1998; 60:431-60.
118. Larsson S, Jones H A, Goransson O, Degerman E, Holm C. Parathyroid hormone induces adipocyte lipolysis via PKA-mediated phosphorylation of hormone-sensitive lipase. Cell Signal. 2016; 28(3): 204-13.
119. Kovanicova Z, Kurdiova T, Balaz M, Stefanicka P, Varga L, Kulterer O C, et al. Cold Exposure Distinctively Modulates Parathyroid and Thyroid Hormones in Cold-Acclimatized and Non-Acclimatized Humans. Endocrinology. 2020; 161(7).
120. Mattsson C L, Csikasz R I, Chernogubova E, Yamamoto D L, Hogberg H T, Amri E Z, et al. beta (1)-Adrenergic receptors increase UCP1 in human MADS brown adipocytes and rescue cold-acclimated beta (3)-adrenergic receptor-knockout mice via nonshivering thermogenesis. Am J Physiol Endocrinol Metab. 2011; 301(6): E1108-18.
121. Arner P, Bernard S, Salehpour M, Possnert G, Liebl J, Steier P, et al. Dynamics of human adipose lipid turnover in health and metabolic disease. Nature. 2011; 478(7367): 110-3.
122. Arner P, Andersson D P, Backdahl J, Dahlman I, Ryden M. Weight Gain and Impaired Glucose Metabolism in Women Are Predicted by Inefficient Subcutaneous Fat Cell Lipolysis. Cell Metab. 2018; 28(1): 45-54 e3.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A composition comprising a therapeutically effective amount of at least one flavanone and a therapeutically effective amount of at least one flavonol.

2. The composition of claim 1, further comprising a therapeutically effective amount of at least one carotenoid.

3. The composition of claim 1, further comprising a sufficient amount of a pharmaceutically acceptable carrier.

4. The method of claim 1, wherein the composition comprises about 150 mg to about 900 mg of at least one flavanone.

5. The method of claim 1, wherein the composition comprises about 1 mg to about 100 mg of at least one flavonol.

6. The method of claim 1, wherein the composition comprises about 1 mg to about 12 mg of at least one carotenoid.

7. The composition of claim 1, wherein the flavanone is naringenin.

8. The composition of claim 1, wherein the flavonol is myricetin

9. The composition of claim 1, wherein the at least one carotenoid is selected from the group consisting of beta carotene, lycopene, or lutein.

10. The composition of claim 1, wherein the at least one carotenoid is beta carotene.

11. The composition of claim 1, wherein the flavanone is naringenin, wherein the flavonol is myricetin, and wherein the carotenoid is beta carotene.

12. The composition of claim 1, wherein the composition further comprises one or more additional active agents.

13. The composition of claim 1, wherein the additional active agent comprises an anti-obesity agent.

14. The composition of claim 1 or claim 2, wherein the composition is provided as an injectable solution, an oral dose, a topical cream, a topical gel, or a medical food.

15. A method for local fat reduction, the method comprising administering to a site on a subject a therapeutically effective amount of the composition of claim 1 or claim 2.

16. A method for treating a subject afflicted with a metabolic disorder, the method comprising administering to a subject a therapeutically effective amount of the composition of claim 1 or claim 2.

17. The method of claim 16, wherein the metabolic disorder comprises an insulin resistance disease.

18. The method of claim 17, wherein the insulin resistance disease comprises type 2 diabetes, polycystic ovarian syndrome, nonalcoholic steatohepatitis, or metabolic syndrome.

19. The method of claim 16, wherein the metabolic disorder comprises obesity.
